(19) United States
(12) Patent Application Publication
(10) Pub. No.: US 2007/0231298 A1
(43) Pub. Date: Oct. 4, 2007

(54) Cytokine-expressing cancer immunotherapy combinations

(75) Inventors: Betty Li, San Francisco, CA (US); Thomas Du, Newark, CA (US); Karin Jooss, Bellevue, WA (US)

Correspondence Address: DLA PIPER US LLP, 153 TOWNSEND STREET, SUITE 800, SAN FRANCISCO, CA, 94107-1957, US

(73) Assignee: Cell Genesys, Inc., South San Francisco, CA (US)

(21) Appl. No.: 11/728,660

(22) Filed: Mar. 27, 2007

Related U.S. Application Data
(60) Provisional application No. 60/788,216, filed on Mar. 31, 2006.

(51) Int. Cl. A61K 38/20; A61N 5/00; A61K 31/517

(52) U.S. Cl. 424/85.1; 514/266.4; 600/1

(57) ABSTRACT

The present invention in all of its associated aspects provides improved methods and compositions for treating cancer in a mammal based on the sequential administration of the combination of a cytokine-expressing cancer immunotherapy composition and at least one tyrosine kinase inhibitor, wherein administration of the combination results in enhanced therapeutic efficacy relative to administration of the cytokine-expressing cancer immunotherapy composition or at least one tyrosine kinase inhibitor as a monotherapy.

This application claims the priority benefit of U.S. Provisional Patent Application No. 60/788,216, filed Mar. 31, 2006. The priority application is expressly incorporated by reference herein in its entirety.

### BACKGROUND OF THE INVENTION

1. Field of The Invention

The present invention relates to a method of altering an individual's immune response to a target cancer antigen or antigens. More particularly, the invention is concerned with the combination of a cytokine-expressing cancer immunotherapy composition and at least one tyrosine-kinase inhibitor and methods of sequentially administering a cytokine-expressing cancer immunotherapy composition in combination with at least one tyrosine-kinase inhibitor.

2. Background Of The Invention

The immune system plays a critical role in the pathogenesis of a wide variety of cancers. When cancers progress, it is widely believed that the immune system either fails to respond sufficiently or fails to respond appropriately, allowing cancer cells to grow. Currently, standard medical treatments for cancer including chemotherapy, surgery, radiation therapy and cellular therapy have clear limitations with regard to both efficacy and toxicity. To date, these approaches have met with varying degrees of success dependent upon the type of cancer, general health of the patient, stage of disease at the time of diagnosis, etc. Improved strategies that combine specific manipulation of the immune response to cancer in combination with standard medical treatments may provide a means for enhanced efficacy and decreased toxicity.

The use of autologous cancer cells as vaccines to augment anti-tumor immunity has been explored for some time (Oettgen et al., “The History of Cancer Immunotherapy”, In: Biologic Therapy of Cancer, Devita et al. (eds.) J. Lippincot Co., pp87-199, 1991). However, due to the weak immunogenicity of many cancers, down regulation of MHC molecules, the lack of adequate costimulatory molecule expression and secretion of immuno-inhibitory cytokines by cancer cells, the response to such vaccines has not resulted in long term efficacy. See, e.g., Armstrong T D and Jaffee E M, Surg Oncol Clin N Am. 11(3):681-96, 2002 and Bodey B et al., Anticancer Res 20(4):2665-76, 2000.

Numerous cytokines have been shown to play a role in regulation of the immune response to tumors. For example, U.S. Pat. No. 5,098,702 describes using combinations of TNF, IL-2 and IFN-beta in synergistically effective amounts to combat existing tumors. U.S. Pat. Nos. 5,078,996, 5,637,483 and 5,904,920 describe the use of GM-CSF for treatment of tumors. However, direct administration of cytokines for cancer therapy may not be practical, as they are often systemically toxic. (See, for example, Asher et al., J. Immunol. 146: 3227-3234, 1991 and Havell et al., J. Exp. Med. 167: 1067-1085, 1988.)

An expansion of this approach involves the use of genetically modified tumor cells which express cytokines locally at the vaccine site. Activity has been demonstrated in tumor models using a variety of immunomodulatory cytokines, including IL-4, IL-2, TNF-alpha, G-CSF, IL-7, IL-6 and GM-CSF, as described in Golumbeck PT et al., Science 254:13-716, 1991; Gansbacher B et al., J. Exp. Med. 172:1217-1224, 1990; Fearon E R et al., Cell 60:397-403, 1990; Gansbacher B et al., Cancer Res. 50:7820-25, 1990; Teng M et al., PNAS 88:3535-3539, 1991; Columbo M P et al., J. Exp. Med. 174:1291-1298, 1991; Aoki et al., Proc Natl Acad Sci U S A. 89(9):3850-4, 1992; Porgador A, et al., Nat Immun. 13(2-3):113-30, 1994; Dranoff G et al., PNAS 90:3539-3543, 1993; Lee C T et al., Human Gene Therapy 8:187-193, 1997; Nagai E et al., Cancer Immunol. Immunother. 47:2-80, 1998 and Chang A et al., Human Gene Therapy 11:839-850, 2000, respectively.

Clinical trials employing GM-CSF-expressing autologous or allogeneic cancer immunotherapy (GVAX®) have commenced for treatment of prostate cancer, melanoma, lung cancer, pancreatic cancer, renal cancer, and multiple myeloma (Dummer R., Curr Opin Investig Drugs 2(6):844-8, 2001; Simons J et al., Cancer Res. 15;59(20):5160-8, 1999; Soiffer R et al., PNAS 95:13141-13146, 1998; Simons J et al., Cancer Res. 15; 57:1537-1546, 1997; Jaffee E et al., J. Clin Oncol. 19:145-156, 2001; and Salgia R et al., J. Clin Oncol. 21:624-630, 2003).

In yet another approach, autologous tumor cells were genetically altered to produce a costimulatory molecule, such as B7-1 or allogeneic histocompatibility antigens (Salvadori et al. Hum. Gene Ther. 6:1299-1306, 1995 and Plaksin et al. Int. J. Cancer 59:796-801, 1994). While the use of genetically modified tumor cells has met with success in treatment of some forms of cancer, there remains a need for improved treatment regimens with greater potency/efficacy and less side effects than the therapies currently in use.

### SUMMARY OF THE INVENTION

The invention provides improved compositions, combinations and methods for the treatment of cancer in a mammal, typically a human, by administering a cytokine-expressing cancer immunotherapy composition in combination with at least one tyrosine kinase inhibitor to a subject with cancer; wherein administration of the combination to the subject results in enhanced therapeutic efficacy relative to administration of the cytokine-expressing cancer immunotherapy or the at least one tyrosine kinase inhibitor alone.

Administration of a cytokine-expressing cancer immunotherapy composition in combination with at least one tyrosine kinase inhibitor results in enhanced immunotherapeutic potency, i.e., an increase in the number and/or proliferation of activated T-cells is detected following administration of the cytokine-expressing cancer immunotherapy composition and the at least one tyrosine kinase inhibitor relative to administration of either the cytokine-expressing cancer immunotherapy composition or the tyrosine kinase inhibitor alone. In a preferred embodiment, the cancer immunotherapy is administered and sufficient time is allowed for tumor antigen specific T-cell priming and activation to occur, whereby subsequent administration of the tyrosine kinase inhibitor leads to enhanced T-cell proliferation and expansion thereby enhancing the therapeutic efficacy of the cancer immunotherapy.

In one aspect of the invention, the cytokine expressing cancer immunotherapy expresses GM-CSF.

In another aspect of the invention, the cytokine-expressing cancer immunotherapy combination comprises cells that are autologous, allogeneic or bystander cells.

In a further aspect of the invention, a population of autologous, allogeneic or bystander cells are genetically modified to produce an effective amount of a cytokine, e.g. GM-CSF.

In particular embodiments, the at least one tyrosine kinase inhibitor is an anilinoquinazoline. In preferred embodiments, the anilinoquinazoline is an inhibitor of Epidermal Growth Factor Receptor (EGFR) activity (erbB2 kinase) and is selected from the group consisting of genfitinib (Iressa) and erolotinib (Tarceva). In other embodiments, the at least one tyrosine kinase inhibitor is an inhibitor of bcr-abl tyrosine kinase activity, preferably imatinib (Gleevec).

The autologous, allogeneic or bystander cells are rendered proliferation incompetent by irradiation prior to administration to the subject.

The cytokine-expressing cancer immunotherapy is typically administered subcutaneously or intratumorally. The at least one tyrosine kinase inhibitor may be administered prior to, at the same time as, or following administration of the cytokine-expressing cancer immunotherapy component of the combination. In a preferred embodiment, the tyrosine kinase inhibitor is an inhibitor of Epidermal Growth Factor Receptor (EGFR) activity (erbB2 kinase), more preferably an anilinoazoquinoline, and is administered to the subject about 4 days, 7 days, 10 days or 14 days following administration of the cytokine-expressing cancer immunotherapy component of the combination to enhance the proliferation of activated cytotoxic T-cells thereby enhancing the efficacy of the cytokine-expressing cancer immunotherapy.

The invention further provides compositions and kits comprising cytokine-expressing cancer immunotherapy combinations for use according to the description provided herein.

### BRIEF DESCRIPTION OF THE DRAWINGS

FIG. 1A is a Kaplan-Meir survival graph of the results of a study in C57Bl/6 mice indicating prior or concurrent administration of a cytokine-expressing cancer immunotherapy and a tyrosine kinase inhibitor results in decreased survival in a basic prevention model. In this model, mice were pretreated daily by oral gavage with 200 mg/kg of genfitinib (Iressa) or erolotinib (Tarceva) starting on Day-11. On Day-7, mice were immunized (subcutaneously; SC) with 1×10⁶ irradiated B16F10 tumor cells engineered to express GM-CSF. Seven days later, mice were challenged (SC) with 1×10⁶ live B16F10 tumor cells and followed for tumor development and survival. The data shown are for control animals (HBSS) with a Mean Survival Time (MST) of 20 days; cytokine-expressing immunotherapy plus genfitinib (Iressa) with a MST of 42 days (B16.GM+Iressa); cytokine-expressing immunotherapy plus erolotinib (Tarceva) with a MST of 31 days (B16.GM+Tarceva); and cytokine-expressing immunotherapy alone.

FIG. 1B is a Kaplan-Meir survival graph of the results of a study in C57Bl/6 mice indicating administration of a cytokine-expressing cancer immunotherapy prior to administering a tyrosine kinase inhibitor results in increased survival in a basic treatment model. In this model, mice were inoculated (SC) with 2×10⁵ live B16F10 tumor cells mice on Day 0 and immunized (SC) with 1×10⁶ irradiated B16F10 tumor cells engineered to express GM-CSF on Days 3 and 17. On Day 15, mice were treated daily by oral gavage with 200 mg/kg of genfitinib (Iressa) or erolotinib (Tarceva), and followed for tumor development and survival.

FIG. 2A is a schematic graph of a study in the B16F10 melanoma model indicating that tyrosine kinase inhibitors inhibit the priming of naïve T-cells but increase the number of activated T-cells. C57Bl6 mice were inoculated with 5×10⁵ B16F10 tumor cells transduced to express ovalbumin as a surrogate antigen on Day 0. One day later, mice began receiving daily by gavage 200 mg/kg of genfitinib (B16.GM+Iressa) or erolotinib (B16.GM+Tarceva). Three days later, mice were immunized with 1×10⁶ irradiated GM-CSF-secreting B16F10 cells engineered to express ovalbumin. Fourteen days later, mice were sacrificed and the spleens were removed and evaluated for induction of a primary T-cell response by quantifying the number of IFN-gamma-secreting T-cells per 5×10⁵ splenocytes when stimulated with an ovalbumin-specific peptide.

FIG. 2B is a schematic graph of a study in the B16F10 melanoma model indicating that tyrosine kinase inhibitors block priming of naïve T-cells but slightly increase the number of activated T-cells. C57Bl6 mice were inoculated with 5×10⁵ B16F10 tumor cells transduced to express ovalbumin as a surrogate antigen on Day 0. On Days 3 and 17, mice were immunized with 1×10⁶ irradiated GM-CSF-secreting B16F10 cells engineered to express ovalbumin. On Day 15, mice began receiving daily by gavage 200 mg/kg of genfitinib (Iressa) or erolotinib (Tarceva). Mice were sacrificed on Day 31 and the spleens were removed and evaluated for induction of a primary T-cell response by quantifying the number of IFN-gamma-secreting T-cells per 5×10⁵ splenocytes when stimulated using an ovalbumin-specific peptide.

FIGS. 3A and 3B illustrate the results of a study demonstrating that tyrosine kinase inhibitors block murine and human naïve T-cell activation. C57Bl/6 mouse lymphocytes or human PBMCs were stimulated using anti-CD3 (FIG. 3A) or ConA (FIG. 3B), respectively. For mouse T cell proliferation, equal numbers of mouse lymphocytes and irradiated antigen presenting cells (1.5×10⁶ cells) were co-cultured for 72 hours in the presence of serially diluted tyrosine kinase inhibitors (TKI), genfitinib (Iressa) or erolotinib (Tarceva), at concentrations ranging from 100 mg/ml to 100 pg/ml. Human T-cell proliferation assay was conducted similarly with 1.5×10⁶ PBMCs co-cultured with 1.5×10⁶ irradiated APCs for 72 hours in the presence of serially diluted genfitinib (Iressa) or erolotinib (Tarceva) at concentrations 100 mg/ml to 100 pg/ml. Cell proliferations were measured by the addition of 1 mCi 3H-thymidine for the last 6 hours of culture. Cells were harvested and counted using a beta counter. Percent inhibition was determined relative to cells alone (no inhibition). Shown are C57BL/6 lymphocytes stimulated with anti-CD3 antibody (FIG. 3A) Human PBMC stimulated with ConA (FIG. 3B).

FIG. 4 illustrates the results of a study demonstrating that tyrosine kinase inhibitors inhibit phosphorylation of tyrosine kinases involved in T-cell activation. Nutrient-starved human T cell clones (Jurkat cells) were stimulated using human anti-CD3 antibodies in the presence of vehicle control (DMSO) or 0, 12.5, or 50 mg/nil of genfitinib (Iressa) or erolotinib (Tarceva). Cells were lysed and immunoprecipitated using a human anti-Pyk2, anti-Zap-70 or anti-Lck antibody. Proteins were boiled, separated by electrophoresis and transferred to nitrocellulose membranes. Membranes were immunoblotted using a human anti-phospho-Pyk2, anti-phospho-Zap-70 or anti-phospho-Lck antibody. Shown is the Western blot evaluating the presence of phosphorylated and non-phosphorrylated tyrosine kinases of stimulated Jurkat cells when co-incubated with a dose titration of genfitinib (Iressa) or erolotinib (Tarceva).

FIG. 5 illustrates the results of a study demonstrating that tyrosine kinase inhibitors augment the expansion of adoptively transferred transgenic T cells upon immunization with cytokine-expressing cancer immunotherapy expressing ovalbumin. On day-7, indicated C57BL/6 recipient mice received daily oral gavages of 200 mg/kg genfitinib (Iressa) or erolotinib (Tarceva) or vehicle only for 20 days. On day-2, 4×10⁶ splenocytes from OT-1 transgenic mice were adoptively transferred into recipient mice. On day 0, mice were challenged with 2×10⁵ live B16F10 cells transduced to express the surrogate antigen, ovalbumin and subsequently immunized with 1×10⁶ irradiated GM-CSF-secreting B16F10 cells transduced to express ovalbumin (GM.ova) on day 3. On day 9, spleens and lymph nodes were harvested, double stained with OT-1-specific tetramers and CD8-specific antibody and evaluated by FACs analysis. Shown is the absolute number of ovalbumin-specific CD8 T cells in spleen and lymph nodes.

FIGS. 6A and 6B provide the structure of an exemplary class of compounds comprising the “anilinoquinazoline” generic core formula. In FIG. 6B, R1, R2, R3 and R4 are members selected from the group consisting of substituted and unsubstituted alkyl, substituted and unsubstituted alkenyl, substituted and unsubstituted alkynyl, substituted and unsubstituted aryl, substituted and unsubstituted cycloalkyl, substituted and unsubstituted cycloalkylheteroalkyl, substituted and unsubstituted arylalkyl, substituted and unsubstituted arylheteroalkyl and combinations thereof, wherein said cycloalkyl portions are monocyclic or polycyclic; hydrogen, OR², C(O)NHR², —C(O)NHS(O)₂R², —NHS(O)₂R², —OC₂—C₄alkyl-C(O)OR², —C(O)R², —C(O)OR² and carboxylic acid analogs, wherein R² is a member selected from the group consisting of hydrogen, , substituted and unsubstituted alkenyl, substituted and unsubstituted alkynyl, substituted and unsubstituted aryl, substituted and unsubstituted cycloalkyl, substituted and unsubstituted cycloalkylheteroalkyl, substituted and unsubstituted arylalkyl, substituted and unsubstituted arylheteroalkyl and combinations thereof. In FIG. 6C, R1 is a halogen or a substituted and unsubstituted alkynyl group, R2 is a halogen, and R3 is a substituted and unsubstituted alkyl or morpholinoalkyl group.

### DETAILED DESCRIPTION

The present invention represents improved cancer immunotherapies for the treatment of cancer in that the compositions and methods described herein comprise at least two components that when administered appropriately act in concert to effect an improved therapeutic outcome for the patient under treatment.

Definitions

Unless otherwise indicated, all terms used herein have the same meaning as they would to one skilled in the art and the practice of the present invention will employ, unless otherwise indicated, conventional techniques of cell biology, molecular biology (including recombinant techniques), microbiology, biochemistry and immunology, which are known to those of skill in the art. Such techniques are explained fully in the literature, such as, “Molecular Cloning: A Laboratory Manual”, second edition (Sambrook et al., 1989); “Oligonucleotide Synthesis” (M. J. Gait; ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991).

The terms “regulating the immune response” or “modulating the immune response” as used herein refers to any alteration in a cell of the immune system or any alteration in the activity of a cell involved in the immune response. Such regulation or modulation includes an increase or decrease in the number of various cell types, an increase or decrease in the activity of these cells, or any other changes which can occur within the immune system. Cells involved in the immune response include, but are hot limited to, T lymphocytes, B lymphocytes, natural killer (NK) cells, macrophages, eosinophils, mast cells, dendritic cells and neutrophils. In some cases, “regulating” or “modulating” the immune response means the immune response is stimulated or enhanced and in other cases “regulating” or “modulating” the immune response means suppression of the immune system. Stimulation of the immune system may include memory responses and/or future protection against subsequent antigen challenge.

The terms “tyrosine kinase inhibitor”, “at least one tyrosine kinase inhibitor” and the like as used herein refer to any molecule that inhibits the activity of a cellular tyrosine kinase. In one aspect, the at least one tyrosine kinase inhibitor is an anilinoquinazoline. In preferred embodiments, the anilinoquinazoline is an inhibitor of Epidermal Growth Factor Receptor (EGFR) activity (erbB2 kinase) and is selected from the group consisting of genfitinib (Iressa) and erolotinib (Tarceva). Other small molecule tyrosine kinase inhibitors that inhibit EGFR activity include 3-cyanoquinolines (e.g., EKB-569), pyrrlopyrimidines (e.g., PKI-166), and 6-thiazolylquinazolines (e.g., GW572016). In other embodiments, the at least one tyrosine kinase inhibitor is an inhibitor of bcr-abl tyrosine kinase activity, preferably imatinib (Gleevec), or the Flt-3 tyrosine kinase. For a review of tyrosine kinase inhibitors see, e.g., Traxler Expert Opin. Ther. Targets 7:215-234 (2003).

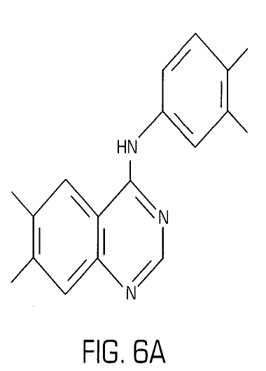

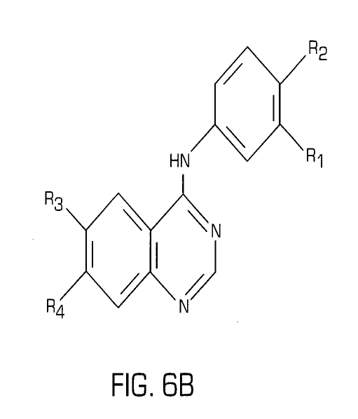

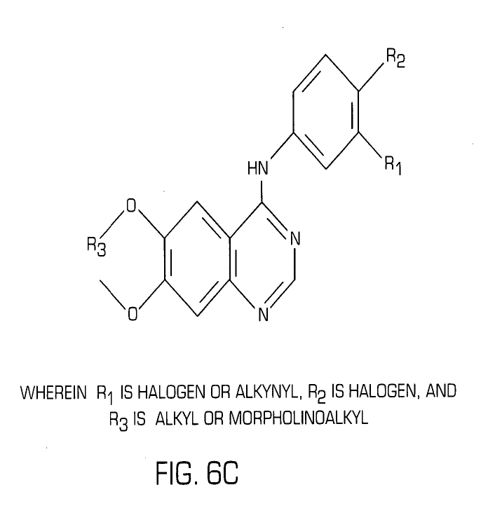

The term “anilinoquinazolines” refers to a class of compounds comprising the following generic core formula, shown in FIGS. 6A and 6B, and pharmaceutical compositions comprising the same.

The term “substituted” as used herein with respect to the anilinoquinazoline compounds shown in FIGS. 6A and B refers to the replacement of an atom or a group of atoms of a compound with another atom or group of atoms. For example, an atom or a group of atoms may be substituted with one or more of the following substituents or groups: halo, cyano, nitro, alkyl, alkylamino, hydroxyalkyl, haloalkyl, carboxyl, hydroxyl, alkoxy, alkoxyalkoxy, haloalkoxy, thioalkyl, aryl, aryloxy, cycloalkyl, cycloalkylalkyl, aryl, heteroaryl optionally substituted with 1 or more, preferably 1 to 3, substituents selected from halo, halo alkyl and alkyl, aralkyl, heteroaralkyl, alkenyl containing 1 to 2 double bonds, alkynyl containing 1 to 2 triple bonds, alk(en)(yn)yl groups, halo, cyano, hydroxy, haloalkyl and polyhaloalkyl, preferably halo lower alkyl, especially trifluoromethyl, formyl, alkylcarbonyl, arylcarbonyl that is optionally substituted with 1 or more, preferably 1 to 3, substituents selected from halo, halo alkyl and alkyl, heteroarylcarbonyl, carboxy, alkoxycarbonyl, aryloxycarbonyl, aminocarbonyl, alkylaminocarbonyl, dialkylaminocarbonyl, arylaminocarbonyl, diarylaminocarbonyl, aralkylaminocarbonyl, alkoxy, aryloxy, perfluoroalkoxy, alkenyloxy, alkynyloxy, arylalkoxy, aminoalkyl, alkylaminoalkyl, dialkylaminoalkyl, arylaminoalkyl, amino, alkylamino, dialkylamino, arylamino, alkylarylamino, alkylcarbonylamino, arylcarbonylamino, azido, nitro, mercapto, alkylthio, arylthio, perfluoroalkylthio, thiocyano, isothiocyano, alkylsulfinyl, alkylsulfonyl, arylsulfinyl, arylsulfonyl, aminosulfonyl, alkylaminosulfonyl, dialkylaminosulfonyl and arylaminosulfonyl. When the term “substituted” appears prior to a list of possible substituted groups, it is intended that the term apply to every member of that group.

The terms “erolotinib”, “Tarceva™” and the like, as used herein refer to the compound with the chemical name: N-(3-ethynylphenyl)-6,7-bis(2-methoxy)-4-quinazolinamine, and pharmaceutical compositions comprising the same.

The terms “genfitinib”, “Iressa” and the like, as used herein refer to the compound with the chemical name: 4-quinazolinamine, N-(3-chloro-4-fluorophenyl)-7-methoxy-6-[3-4-morpholin) propoxyl], and pharmaceutical compositions comprising the same.

The terms “imatinib”, “Gleevec” and the like as used herein refer to the compound with the chemical name: 4-[(4-methyl-1-piperazinyl)methyl]-N-[4-methyl-3-[[4-(3-pyridinyl)-2-pyriminidinyl]amino]-phenyl]benzamide methanesulfonate, and pharmaceutical compositions thereof.

The term “cytokine” or “cytokines” as used herein refers to the general class of biological molecules which effect/affect cells of the immune system. The definition is meant to include, but is not limited to, those biological molecules that act locally or may circulate in the blood, and which, when used in the compositions or methods of the present invention serve to regulate or modulate an individual's immune response to cancer. Exemplary cytokines for use in practicing the invention include but are not limited to IFN-alpha, IFN-beta, and IFN-gamma, interleukins (e.g., IL-1 to IL-29, in particular, IL-2, IL-7, IL-12, IL-15 and IL-18), tumor necrosis factors (e.g., TNF-alpha and TNF-beta), erythropoietin (EPO), MIP3a, ICAM, macrophage colony stimulating factor (M-CSF), granulocyte colony stimulating factor (G-CSF) and granulocyte-macrophage colony stimulating factor (GM-CSF).

The term “cytokine-expressing cancer immunotherapy” as used herein refers to a composition comprising a population of cells that has been genetically modified to express a cytokine, e.g., GM-CSF, and that is administered to a patient as part of a cancer treatment regimen. The cells of such a “cytokine-expressing cancer immunotherapy” comprise a cytokine-encoding DNA sequence operably linked to expression and control elements such that the cytokine is expressed by the cells. The cells of the “cytokine-expressing cancer immunotherapy” are typically tumor cells and may be autologous or allogeneic to the patient undergoing treatment and or may be “bystander cells” that are mixed with tumor cells taken from the patient. A GM-CSF-expressing “cytokine-expressing cancer immunotherapy” may be referred to herein as “GVAX®”.

The term “operably linked” as used herein relative to a recombinant DNA construct or vector means nucleotide components of the recombinant DNA construct or vector are directly linked to one another for operative control of a selected coding sequence. Generally, “operably linked” DNA sequences are contiguous, and, in the case of a secretory leader, contiguous and in reading frame, however, some sequences, e.g., enhancers do not have to be contiguous.

The “vector” may be a DNA molecule such as a plasmid, virus or other vehicle, which contains one or more heterologous or recombinant DNA sequences, e.g., a nucleic acid sequence encoding a cytokine under the control of a functional promoter and in some cases further including an enhancer that is capable of functioning as a vector, as understood by those of ordinary skill in the art. An appropriate viral vector includes, but is not limited to, a retrovirus, a lentivirus, an adenovirus (AV), an adeno-associated virus (AAV), a simian virus 40 (SV-40), a bovine papilloma virus, an Epstein-Barr virus, a herpes virus, a vaccinia virus, a Moloney murine leukemia virus, a Harvey murine sarcoma virus, a murine mammary tumor virus, and a Rous sarcoma virus. Non-viral vectors are also included within the scope of the invention.

As used herein, the term “gene” or “coding sequence” means the nucleic acid sequence which is transcribed (DNA) and translated (mRNA) into a polypeptide in vitro or in vivo when operably linked to appropriate regulatory sequences. A “gene” typically comprises the coding sequence plus any non-coding sequences associated with the gene (e.g., regulatory sequences) and hence may or may not include regions preceding and following the coding region, e. g. 5′ untranslated (5′UTR) or “leader” sequences and 3′ UTR or “trailer” sequences, as well as intervening sequences (introns) between individual coding segments (exons). In contrast, a “coding sequence” does not include non-coding DNA.

The terms “gene-modified” and “genetically-modified” are used herein with reference to a cell or population of cells wherein a nucleic acid sequence has been introduced into the cell or population of cells. The nucleic acid sequence may be heterologous to the cell(s), or it may be an additional copy or improved version of a nucleic acid sequence already present in the cell(s). The cell(s) may be genetically-modified by physical or chemical methods or by the use of recombinant viruses. Chemical and physical methods such as calcium phosphate, electroporation and pressure mediated transfer of genetic material into cells are often used. Several recombinant viral vectors which find utility in effective delivery of genes into mammalian cells include, for example, retroviral vectors, adenovirus vectors, adenovirus-associated vectors (AAV), herpes virus vectors, pox virus vectors. In addition, non-viral means of introduction, for example, naked DNA delivered via liposomes, receptor-mediated delivery, calcium phosphate transfection, electroporation, particle bombardment (gene gun), or pressure-mediated delivery may also be employed to introduce a nucleic acid sequence into a cell or population of cells to render them “gene-modified” or “genetically-modified

Reference to a vector or other DNA sequences as “recombinant” merely acknowledges the operable linkage of DNA sequences which are not typically operably linked as isolated from or found in nature. A “promoter” is a DNA sequence that directs the binding of RNA polymerase and thereby promotes RNA synthesis. “Enhancers” are cis-acting elements that stimulate or inhibit transcription of adjacent genes. An enhancer that inhibits transcription also is termed a “silencer”. Enhancers can function (i.e. be operably linked to a coding sequence) in either orientation, over distances of up to several kilobase pairs (kb) from the coding sequence and from a position downstream of a transcribed region. Regulatory (expression/control) sequences are operatively linked to a nucleic acid coding sequence when the expression/control sequences regulate the transcription and, as appropriate, translation of the nucleic acid sequence. Thus expression/control sequences can include promoters, enhancers, transcription terminators, a start codon (i.e., ATG) in front of the coding sequined, splicing signal for introns and stop codons.

As used herein, the terms “tumor” and “cancer” refer to a cell that exhibits a loss of growth control and forms unusually large clones of cells. Tumor or cancer cells generally have lost contact inhibition and may be invasive and/or have the ability to metastasize.

“Cancer” as used herein includes cancer localized in tumors, as well as cancer not localized in tumors, such as, for instance, cancer cells that expand from a local tumor by invasion (i.e., metastasis). The invention finds utility in the treatment of any form of cancer, including, but not limited to, cancer of the bladder, breast, colon, kidney, liver, lung, ovary, cervix, pancreas, rectum, prostate, stomach, epidermis; a hematopoietic tumor of lymphoid or myeloid lineage; a tumor of mesenchymal origin such as a fibrosarcoma or rhabdomyosarcoma; other tumor types such as melanoma, teratocarcinoma, neuroblastoma, glioma, adenocarcinoma and non-small lung cell carcinoma.

The term “antigen from a tumor cell” and “tumor antigen” and “tumor cell antigen” may be used interchangeably herein and refer to any protein, carbohydrate or other component derived from or expressed by a tumor cell which is capable of eliciting an immune response. The definition is meant to include, but is not limited to, whole tumor cells that express all of the tumor-associated antigens, tumor cell fragments, plasma membranes taken from a tumor cell, proteins purified from the cell surface or membrane of a tumor cell, or unique carbohydrate moieties associated with the cell surface of a tumor cell. The definition also includes those antigens from the surface of the cell which require special treatment of the cells to access.

The term “systemic immune response” as used herein means an immune response which is not localized, but affects the individual as a whole.

The term “gene therapy” as used herein means the treatment or prevention of cancer by means of ex vivo or in vivo delivery, through viral or non-viral vectors, of compositions containing a recombinant genetic material.

The term “ex vivo” delivery as used herein means the introduction, outside of the body of a human, of compositions containing a genetic material into a cell, tissue, organoid, organ, or the like, followed by the administration of cell, tissue, organoid, organ, or the like which contains such introduced compositions into the body of the same (autologous) or a different (allogeneic) human, without limitation as to the formulation, site or route of administration.

The terms “inactivated cells”, “non-dividing cells” and “non-replicating cells” may be used interchangeably herein and refer to cells that have been treated rendering them proliferation incompetent, e.g., by irradiation. Such treatment results in cells that are unable to undergo mitosis, but retain the capability to express proteins such as cytokines or other cancer therapeutic agents. Typically a minimum dose of about 3500 rads is sufficient, although doses up to about 30,000 rads are acceptable. Effective doses include, but are not limited to 5000 to 10000 rads. Numerous methods of inactivating cells, such as treatment with Mitomycin C, are known in the art. Any method of inactivation which renders cells incapable of cell division, but allows the cells to retain the ability to express proteins is included within the scope of the present invention.

As used herein “treatment” of an individual or a cell is any type of intervention used in an attempt to alter the natural course of the individual or cell. Treatment includes, but is not limited to, administration of e.g., a cytokine-expressing cancer immunotherapy and at least one tyrosine kinase inhibitor, and may be performed either prophylactically or subsequent to diagnosis as part of a primary or follow-up therapeutic regimen.

The term “administering” as used herein refers to the physical introduction of a composition comprising a cytokine-expressing cancer immunotherapy and at least one tyrosine kinase inhibitor to a patient with cancer. Any and all methods of introduction are contemplated according to the invention, the method is not dependent on any particular means of introduction and is not to be so construed. Means of introduction are well-known to those skilled in the art, examples of which are provided herein.

The term “co-administering” as used herein means a process whereby the combination of a cytokine-expressing cancer immunotherapy and at least one tyrosine kinase inhibitor is administered to the same patient. The cytokine-expressing cancer immunotherapy and at least one tyrosine kinase inhibitor may be administered simultaneously, at essentially the same time, or sequentially. If administration takes place sequentially, the cytokine-expressing cancer immunotherapy may be administered before or after a given at least one tyrosine kinase inhibitor. The cytokine-expressing cancer immunotherapy and at least one tyrosine kinase inhibitor need not be administered by means of the same vehicle, the cancer immunotherapy and at least one tyrosine kinase inhibitor may be administered one or more times and the number of administrations of each component of the combination may be the same or different. In addition, the cytokine-expressing cancer immunotherapy and at least one tyrosine kinase inhibitor need not be administered at the same site. In a preferred embodiment, the tyrosine kinase inhibitor is an inhibitor of Epidermal Growth Factor Receptor (EGFR) activity, more preferably is an anilinoazoquinoline, and is administered to the subject about 4 days, 7 days, 10 days or 14 days following administration of the cytokine-expressing cancer immunotherapy component of the combination to enhance the proliferation of activated T-cells thereby enhancing the efficacy of the cytokine-expressing cancer immunotherapy.

The term “therapeutically effective amount” or “therapeutically effective combination” as used herein refers to an amount or dose of a cytokine-expressing cancer immunotherapy together with the amount or dose of an additional agent or treatment that is sufficient to modulate, either by stimulation or suppression, the systemic immune response of an individual. The amount of cytokine-expressing cancer immunotherapy in a given therapeutically effective combination may be different for different individuals, different tumor types and will be dependent upon the one or more additional agents or treatments included in the combination. The “therapeutically effective amount” is determined using procedures routinely employed by those of skill in the art such that an “improved therapeutic outcome” results.

As used herein, the terms “improved therapeutic outcome” and “enhanced therapeutic efficacy”, relative to cancer refers to a slowing or diminution of the growth of cancer cells or a solid tumor, or a reduction in the total number of cancer cells or total tumor burden. An “improved therapeutic outcome” or “enhanced therapeutic efficacy” therefore means there is an improvement in the condition of the patient according to any clinically acceptable criteria, including an increase in time to tumor progression, an increase in life expectancy, or an improvement in quality of life.

The term “reversal of an established tumor” as used herein means the suppression, regression, partial or complete disappearance of a pre-existing tumor. The definition is meant to include any diminution in the size, growth rate, appearance or cellular compositions of a preexisting tumor.

The terms “individual”, “subject” as referred to herein is a vertebrate, preferably a mammal, and typically refers to a human.

Methods and Compositions of the Invention

The methods and compositions of the invention provide an improved therapeutic approach to the treatment of cancer by co-administration of a cytokine-expressing cancer immunotherapy and at least one tyrosine kinase inhibitor to a patient with cancer.

Introduction Of Cytokine Encoding Nucleic Acids Into Cells

In one aspect of the invention, a nucleotide sequence (i.e., a recombinant DNA construct or vector) encoding a cytokine operably linked to a promoter is introduced into a cell or population of cells. Any and all methods of introduction into a cell or population of cells, typically tumor cells, are contemplated according to the invention, the method is not dependent on any particular means of introduction and is not to be so construed. The cytokine-encoding nucleic acid sequence may be introduced into the same or a different population of cells.

Any suitable vector can be employed that is appropriate for introduction of nucleic acids into eukaryotic tumor cells, or more particularly animal tumor cells, such as mammalian, e.g., human, tumor cells. Preferably the vector is compatible with the tumor cell, e.g., is capable of imparting expression of the coding sequence for a cytokine or cancer therapeutic agent, and is stably maintained or relatively stably maintained in the tumor cell. Desirably the vector comprises an origin of replication and the vector may or may not also comprise a “marker” or “selectable marker” function by which the vector can be identified and selected. While any selectable marker can be used, selectable markers for use in such expression vectors are generally known in the art and the choice of the proper selectable marker will depend on the host cell. Examples of selectable marker genes which encode proteins that confer resistance to antibiotics or other toxins include ampicillin, methotrexate, tetracycline, neomycin (Southern and Berg, J., 1982), mycophenolic acid (Mulligan and Berg, 1980), puromycin, zeomycin, hygromycin (Sugden et al., 1985) or G418.

In practicing the methods of the present invention, a vector comprising a nucleotide sequence encoding a cytokine may be transferred to a cell in vitro, preferably a tumor cell, using any of a number of methods which include but are not limited to electroporation, membrane fusion with liposomes, Lipofectamine treatment, high velocity bombardment with DNA-coated microprojectiles, incubation with calcium phosphate-DNA precipitate, DEAE-dextran mediated transfection, infection with modified viral nucleic acids, direct microinjection into single cells, etc. Procedures for the cloning and expression of modified forms of a native protein using recombinant DNA technology are generally known in the art, as described in Ausubel, et al., 1992 and Sambrook, et al., 1989, expressly incorporated by reference, herein.

Recombinant vectors for the production of cancer immunotherapy of the invention provide all the proper transcription, translation and processing signals (e.g., splicing and polyadenylation signals) such that the coding sequence for the cytokine is appropriately transcribed and translated in the tumor cells into which the vector is introduced. The manipulation of such signals to ensure appropriate expression in host cells is within the skill of the ordinary skilled artisan. The coding sequence for the cytokine may be under control of (i.e., operably linked to) it's own native promoter, or a non-native (i.e. heterologous) promoter, including a constitutive promoter, e.g., the cytomegalovirus (CMV) immediate early promoter/enhancer, the Rous sarcoma virus long terminal repeat (RSV-LTR) or the SV-40 promoter.

Alternately, a tissue-specific promoter (a promoter that is preferentially activated in a particular type of tissue and results in expression of a gene product in that tissue) can be used in the vector. Such promoters include but are not limited to a liver specific promoter (III CR, et al., Blood Coagul Fibrinolysis 8 Suppl 2:S23-30, 1997) and the EF-1 alpha promoter (Kim D W et al. Gene. 91(2):217-23, 1990, Guo Z S et al. Gene Ther. 3(9):802-10, 1996; U.S. Pat. Nos. 5,266,491 and 5,225,348, each of which expressly incorporated by reference herein). Inducible promoters also find utility in practicing the methods described herein, such as a promoter containing the tet responsive element (TRE) in the tet-on or tet-off system as described (ClonTech and BASF), the metallothienein promoter which can be upregulated by addition of certain metal salts and rapamycin inducible promoters (Rivera et al., 1996, Nature Med, 2(9):1028-1032; Ye et al., 2000, Science 283: 88-91; Sawyer T K et al., 2002, Mini Rev Med Chem. 2(5):475-88). Large numbers of suitable tissue-specific or regulatable vectors and promoters for use in practicing the current invention are known to those of skill in the art and many are commercially available.

Exemplary vector systems for use in practicing the invention include the retroviral MFG vector, described in U.S. Pat. No. 5,637,483, expressly incorporated by reference herein. Other useful retroviral vectors include pLJ, pEm and [alpha]SGC, described in U.S. Pat. No. 5,637,483 (in particular Example 12), U.S. Pat. Nos. 6,506,604, 5,955,331 and U.S. Ser. No. 09/612808, each of which is expressly incorporated by reference herein.

Further exemplary vector systems for use in practicing the invention include second, third and fourth generation lentiviral vectors, U.S. Pat. Nos. 6,428,953, 5,665,577 and 5,981,276 and WO 00/72686, each of which is expressly incorporated by reference herein.

Additional exemplary vector systems for use in practicing the present invention include adenoviral vectors, described for example in U.S. Pat. No. 5,872,005 and WO 00/72686, each of which is expressly incorporated by reference herein.

Yet another vector system that is preferred in practicing the methods described herein is a recombinant adeno-associated vector (rAAV) system, described for example in WO98/46728, WO 00/72686, Samulski et al., Virol. 63:3822-3828(1989) and U.S. Pat. Nos. 5,436,146, 5,753,500, 6,037,177, 6,040,183 and 6,093,570, each of which is expressly incorporated by reference herein.

Cytokines

Cytokines and combinations of cytokines have been shown to play an important role in the stimulation of the immune system. The term “cytokine” is understood by those of skill in the art, as referring to any immunopotentiating protein (including a modified protein such as a glycoprotein) that enhances or modifies the immune response to a tumor present in the host. The cytokine typically enhances or modifies the immune response by activating or enhancing the activity of cells of the immune system and is not itself immunogenic to the host.

It follows from the results presented herein that a variety of cytokines will find use in the present invention. Exemplary cytokines for use in practicing the invention include but are not limited to IFN-alpha, IFN-beta, and IFN-gamma, interleukins (e.g., IL-1 to IL-29, in particular, IL-2, IL-7, IL-12, IL-15 and IL-18), tumor necrosis factors (e.g., TNF-alpha and TNF-beta), erythropoietin (EPO), MIP3a, macrophage colony stimulating factor (M-CSF), granulocyte colony stimulating factor (G-CSF) and granulocyte-macrophage colony stimulating factor (GM-CSF). The cytokine may be from any source, however, optimally the cytokine is of murine or human origin (a native human or murine cytokine) or is a sequence variant of such a cytokine, so long as the cytokine has a sequence with substantial homology to the human form of the cytokine and exhibits a similar activity on the immune system. It follow that cytokines with substantial homology to the human forms of IFN-alpha, IFN-beta, and IFN-gamma, IL-1 to IL-29, TNF-alpha, TNF-beta, EPO, MIP3a, ICAM, M-CSF, G-CSF and GM-CSF are useful in practicing the invention, so long as the homologous form exhibits the same or a similar effect on the immune system. Proteins that are substantially similar to any particular cytokine, but have relatively minor changes in protein sequence find use in the present invention. It is well known that small alterations in protein sequence may not disturb the functional activity of a protein molecule, and thus proteins can be made that function as cytokines in the present invention but differ slightly from current known or native sequences.

Variant Sequences

Homologues and variants of native human or murine cytokines are included within the scope of the invention. As used herein, the term “sequence identity” means nucleic acid or amino acid sequence identity between two or more aligned sequences and is typically expressed as a percentage (“%”). The term “% homology” is used interchangeably herein with the term “% identity” or “% sequence identity” and refers to the level of nucleic acid or amino acid sequence identity between two or more aligned sequences, when aligned using a sequence alignment program. For example, as used herein, 80% homology means the same thing as 80% sequence identity determined by a defined algorithm, and accordingly a homologue of a given sequence typically has greater than 80% sequence identity over a length of the given sequence. Preferred levels of sequence identity include, but are not limited to, 80, 85, 88, 89, 90, 91, 92, 93, 94, 95, 96, 97, 98 or 99% or more sequence identity to a native cytokine or cancer therapeutic agent amino acid or nucleic acid sequence, as described herein.

Exemplary computer programs that can be used to determine the degree of identity between two sequences include, but are not limited to, the suite of BLAST programs, e.g., BLASTN, BLASTX, TBLASTX, BLASTP and TBLASTN, all of which are publicly available on the Internet. See, also, Altschul, S. F. et al. Mol. Biol. 215:403-410, 1990 and Altschul, S. F. et al. Nucleic Acids Res. 25:3389-3402, 1997, expressly incorporated by reference herein. Sequence searches are typically carried out using the BLASTN program when evaluating a given nucleotide sequence relative to nucleotide sequences in the GenBank DNA Sequences and other public databases. The BLASTX program is preferred for searching nucleotide sequences that have been translated in all reading frames against amino acid sequences in the GenBank Protein Sequences and other public databases. In determining sequence identity, both BLASTN and BLASTX (i.e. version 2.2.5) are run using default parameters of an open gap penalty of 11.0, and an extended gap penalty of 1.0, and utilize the BLOSUM-62 matrix. [See, Altschul, et al., 1997, supra.] A preferred alignment of selected sequences in order to determine “% identity” between two or more sequences, is performed using for example, the CLUSTAL-W program in MacVector version 6.5, operated with default parameters, including an open gap penalty of 10.0, an extended gap penalty of 0.1, and a BLOSUM 30 similarity matrix.

A nucleotide sequence is considered to be “selectively hybridizable” to a reference nucleotide sequence if the two sequences specifically hybridize to one another under moderate to high stringency hybridization and wash conditions. Hybridization conditions are based on the melting temperature (Tm) of the nucleic acid binding complex or probe. For example, “maximum stringency” typically occurs at about TM-5° C. (5° below the Tm of the probe) “high stringency” at about 5-10° below the Tm; “intermediate stringency” at about 10-20° below the Tm of the probe; and “low stringency” at about 20-25° below the Tm. Functionally, maximum stringency conditions may be used to identify sequences having strict identity or near-strict identity with the hybridization probe, while high stringency conditions are used to identify sequences having about 80% or more sequence identity with the probe. An example of high stringency conditions includes hybridization at about 42° C. in 50% formamide, 5× SSC, 5× Denhardt's solution, 0.5% SDS and 100 μg/ml denatured carrier DNA followed by washing two times in 2× SSC and 0.5% SDS at room temperature and two additional times in 0.1× SSC and 0.5% SDS at 42° C. Moderate and high stringency hybridization conditions are well known in the art. See, for example, Sambrook, et al, 1989, Chapters 9 and 11, and in Ausubel, F. M., et al., 1993, (expressly incorporated by reference herein).

One Or More Tyrosine Kinase Inhibitors

As detailed herein, the present invention is directed to a method of improving an individual's immune response to cancer (e.g., a target cancer antigen or antigens) by co-administering a cytokine-expressing cancer immunotherapy (e.g., GM-CSF; GVAX®) and at least one tyrosine kinase inhibitor to a patient with cancer. Tyrosine kinase inhibitors for use in practicing the invention include, but are not limited to, molecules that inhibit the activity of cellular tyrosine kinases, including those that inhibit the activity of EGFR (erbB2 kinase) or bcl-abl tyrosine kinases and combinations thereof (e.g., see Traxler Expert Opin. Ther. Targets 7:215-234 (2003); Baselga and Hammond Oncology 63(suppl 1): 6-16 (2002)).

Cancer Immunotherapy Combinations

Granulocyte-macrophage colony stimulating factor (GM-CSF) is a cytokine produced by fibroblasts, endothelial cells, T cells and macrophages. This cytokine has been shown to induce the growth of hematopoetic cells of granulocyte and macrophage lineages. In addition, it activates the antigen processing and presenting function of dendritic cells, which are the major antigen presenting cells (APC) of the immune system. Results from animal model experiments have convincingly shown that GM-CSF producing tumor cells (i.e. GVAX®) are able to induce an immune response against parental, non-transduced tumor cells.

Autologous and allogeneic cancer cells that have been genetically modified to express a cytokine, e.g., GM-CSF, followed by readministration to a patient for the treatment of cancer are described in U.S. Pat. Nos. 5,637,483, 5,904,920 and 6,350,445, expressly incorporated by reference herein. A form of GM-CSF-expressing genetically modified cancer cells or a “cytokine-expressing cancer immunotherapy” for the treatment of pancreatic cancer is described in U.S. Pat. Nos. 6,033,674 and 5,985,290, expressly incorporated by reference herein. A universal immunomodulatory cytokine-expressing bystander cell line is described in U.S. Pat. No. 6,464,973, expressly incorporated by reference herein. Clinical trials employing GM-CSF-expressing autologous or allogeneic cancer immunotherapy (GVAX®) have been undertaken for treatment of prostate cancer, melanoma, lung cancer, pancreatic cancer, renal cancer, and multiple myeloma, and a number of these trials are currently ongoing, however, the question still remains open as to whether the immune response to GM-CSF alone will be sufficiently powerful to slow or eradicate large or fast growing malignancies.

The present invention provides an improved method of stimulating an immune response to cancer in a mammalian, preferably a human, patient. Desirably, the method effects a systemic immune response, i.e., a T-cell response and/or a B-cell response, to the cancer. The method comprises administering to the patient a cytokine-expressing cancer immunotherapy and at least one tyrosine kinase inhibitor, wherein the cancer immunotherapy comprises cells which express a cancer antigen or various cancer antigens, the cancer antigen/antigens can be one of the antigens of the cancer found in the patient under treatment. The cells are rendered proliferation incompetent, such as by irradiation. Upon administration of the composition, an immune response to the cancer is elicited or enhanced. In one approach, the cancer immunotherapy comprises two or more populations of cells individually modified to express one component of the cancer immunotherapy. In another approach, the cytokine-expressing cancer immunotherapy combination comprises a population of cells that is modified to express a cytokine which is administered in combination with at least one tyrosine kinase inhibitor.

In general, a cytokine-expressing cancer immunotherapy combination for use in practicing the invention comprises tumor cells selected from the group consisting of autologous tumor cells, allogeneic tumor cells and tumor cell lines (i.e., bystander cells).

In some embodiments, the cells of the cytokine-expressing cancer immunotherapy combination are cryopreserved prior to administration. In one aspect of the invention, the cells of the cytokine-expressing cancer immunotherapy combination are administered to the same individual from whom they were derived (autologous). In another aspect of the invention, the cells of the cytokine-expressing cancer immunotherapy combination and the tumor are derived from different individuals (allogeneic or bystander). The invention finds utility in the treatment of any form of tumor or cancer, including, but not limited to, cancer of the bladder, breast, colon, kidney, liver, lung, ovary, cervix, pancreas, rectum, prostate, stomach, epidermis; a hematopoietic tumor of lymphoid or myeloid lineage; a tumor of mesenchymal origin such as a fibrosarcoma or rhabdomyosarcoma; other tumor types such as melanoma, teratocarcinoma, neuroblastoma, glioma, adenocarcinoma and non-small lung cell carcinoma.

In one aspect of the invention, the cells of the cytokine-expressing cancer immunotherapy combination comprises gene-modified cells of one type for the expression of the cytokine and of another different type for expression of the one or more additional cancer therapeutic agents. By way of example, in one approach, the cytokine-expressing cancer immunotherapy (i.e., GVAX®) is provided as an allogeneic or bystander.

In previous studies, a direct comparison of murine tumor cells transduced with various cytokines demonstrated that GM-CSF-secreting tumor cells induced the best overall anti-tumor protection. In one preferred embodiment, the cytokine expressed by the cytokine-expressing cancer immunotherapy of the invention is GM-CSF (generally referred to herein as “GVAX®”). The preferred coding sequence for GM-CSF is the genomic sequence described in Huebner K. et al., Science 230(4731):1282-5, 1985. Alternatively the cDNA form of GM-CSF finds utility in practicing the invention (Cantrell et al., Proc. Natl. Acad. Sci., 82, 6250-6254, 1985).

Prior to administration, the cells of a cytokine-expressing cancer immunotherapy combination of the invention are rendered proliferation incompetent. While a number of means of rendering cells proliferation incompetent are known, irradiation is the preferred method. Preferably, the cytokine-expressing cancer immunotherapy combination is irradiated at a dose of from about 50 to about 200 rads/min, even more preferably, from about 120 to about 140 rads/min prior to administration to the patient. Most importantly, the cells are irradiated with a total radiation dose sufficient to inhibit growth of substantially 100% of the cells, from further proliferation. Thus, desirably the cells are irradiated with a total dose of from about 10,000 to 20,000 rads, optimally, with about 15,000 rads.

Autologous

The use of autologous cytokine-expressing cells in a cancer immunotherpay composition of the invention provides advantages since each patient's tumor expresses a unique set of tumor antigens that can differ from those found on histologically-similar, MHC-matched tumor cells from another patient. See, e.g., Kawakami et al., J. Immunol., 148, 638-643 (1992); Darrow et al., J. Immunol., 142, 3329-3335 (1989); and Hom et al., J. Immunother., 10, 153-164 (1991). In contrast, MHC-matched tumor cells provide the advantage that the patient need not be taken to surgery to obtain a sample of their tumor for immunotherapy vaccine production.

In one preferred aspect, the present invention comprises a method of treating cancer by carrying out the steps of: (a) obtaining tumor cells from a mammal, preferably a human, harboring a tumor; (b) modifying the tumor cells to render them capable of producing a cytokine or an increased level of a cytokine naturally produced by the cells; (c) rendering the modified tumor cells proliferation incompetent; (d) readministering the modified tumor cells to the mammal from which the tumor cells were obtained or to a mammal with the same MHC type as the mammal from which the tumor cells were obtained; and (e) administering a therapeutically effective amount of a tyrosine kinase inhibitor 4 days, 7 days, 10 days or 14 days after readministering the modified tumor cells, whereby administration of the combination to the subject results in enhanced therapeutic efficacy relative to administration of the autologous cytokine-expressing cancer immunotherapy or the at least one tyrosine kinase inhibitor.

Allogeneic

Researchers have sought alternatives to autologous and MHC-matched cells as tumor vaccines, as reviewed by Jaffee et al., Seminars in Oncology, 22, 81-91 (1995). Early tumor vaccine strategies were based on the understanding that the vaccinating tumor cells function as the antigen presenting cells (APCs) and present tumor antigens by way of their MHC class I and II molecules, and directly activate the T cell arm of the immune system. The results of Huang et al. (Science, 264, 961-965, 1994), indicate that professional APCs of the host rather than the vaccinating tumor cells prime the T cell arm of the immune system by secreting cytokine(s) such as GM-CSF such that bone marrow-derived APCs are recruited to the region of the tumor. The bone marrow-derived APCs take up the whole cellular protein of the tumor for processing, and then present the antigenic peptide(s) on their MHC class I and II molecules, thereby priming both the CD4+ and the CD8+ T cell arms of the immune system, resulting in a systemic tumor-specific anti-tumor immune response. These results suggest that it may not be necessary or optimal to use autologous or MHC-matched tumor cells in order to elicit an anti-cancer immune response and that the transfer of allogeneic MHC genes (from a genetically dissimilar individual of the same species) can enhance tumor immunogenicity. More specifically, in certain cases, the rejection of tumors expressing allogeneic MHC class I molecules resulted in enhanced systemic immune responses against subsequent challenge with the unmodified parental tumor, as reviewed in Jaffee et al., supra, and Huang et al., supra.

As described herein, a “tumor cell line” comprises cells that were initially derived from a tumor. Such cells typically are transformed (i.e., exhibit indefinite growth in culture).

In one preferred aspect, the invention provides a method for treating cancer by carrying out the steps of: (a) obtaining a tumor cell line; (b) modifying the tumor cell line to render the cells capable of producing an increased level of a cytokine relative to the unmodified tumor cell line; (c) rendering the modified tumor cell line proliferation incompetent; (d) administering the tumor cell line to a mammalian host having at least one tumor that is the same type of tumor as that from which the tumor cell line was obtained or wherein the tumor cell lien and host tumor express at least one common antigen; and (e) administering a therapeutically effective amount of a tyrosine kinase inhibitor 4 days, 7 days, 10 days or 14 days after readministering the modified tumor cells, whereby administration of the combination to the subject results in enhanced therapeutic efficacy relative to administration of the autologous cytokine-expressing cancer immunotherapy or the at least one tyrosine kinase inhibitor.

The administered tumor cell line is allogeneic and is not MHC-matched to the host. Such allogeneic lines provide the advantage that they can be prepared in advance, characterized, aliquoted in vials containing known numbers of cytokine-expressing cells and stored such that well characterize cells are available for administration to the patient. Methods for the production of gene-modified allogeneic cells are described for example in WO 00/72686A1, expressly incorporated by reference herein.

In one approach to preparing a cytokine-expressing cancer immunotherapy comprising gene-modified allogeneic cells, cytokine is introduced into a cell line that is an allogeneic tumor cell line (i.e., derived from an individual other than the individual being treated). The tumor and/or tumor cell line may be from any form of cancer, including, but not limited to, carcinoma of the bladder, breast, colon, kidney, liver, lung, ovary, cervix, pancreas, rectum, prostate, stomach, epidermis; a hematopoietic tumor of lymphoid or myeloid lineage; a tumor of mesenchymal origin such as a fibrosarcoma or rhabdomyosarcoma; or another tumor, including a melanoma, teratocarcinoma, neuroblastoma, glioma, adenocarcinoma and non-small lung cell carcinoma.

Desirably, the allogeneic cell line expresses GM-CSF in a range from 200-1000 ng/10⁶ cells/24 h. Preferably, the universal bystander cell line expresses at least about 200 ng GM-CSF/10⁶ cells/24 hours.

In practicing the invention, one or more allogeneic cell lines are incubated with an autologous cancer antigen, e.g., an autologous tumor cell (which together comprise an allogeneic cell line composition), then the allogeneic cell line composition is administered to the patient. Typically, the cancer antigen is provided by (on) a cell of the cancer to be treated, i.e., an autologous cancer cell. In such cases, the composition is rendered proliferation-incompetent by irradiation, wherein the allogeneic cells and cancer cells are plated in a tissue culture plate and irradiated at room temperature using a Cs source, as detailed above. The ratio of allogeneic cells to autologous cancer cells in a given administration will vary dependent upon the combination.

Any suitable route of administration can be used to introduce an allogeneic cell line composition into the patient, preferably, the composition is administered subcutaneously or intratumorally.

The use of allogeneic cell lines in practicing present invention provides the therapeutic advantage that, through administration of a cytokine-expressing allogeneic cell line and at least tyrosine kinase inhibitor to a patient with cancer, together with an autologous cancer antigen, paracrine production of an immunomodulatory cytokine, results in an effective immune response to a tumor. This obviates the need to culture and transduce autologous tumor cells for each patient, eliminating the problem of variable and inefficient transduction efficiencies.

Bystander

In one further aspect, the present invention provides a universal immunomodulatory cytokine-expressing bystander cell line. The universal bystander cell line comprises cells which either naturally lack major histocompatibility class I (MHC-I) antigens and major histocompatibility class II (MHC-II) antigens or have been modified so that they lack MHC-I antigens and MHC-II antigens. In one aspect of the invention, a universal bystander cell line is modified by introduction of a vector comprising a nucleotide sequence encoding a cytokine operably linked to a promoter and expression control sequences necessary for expression thereof. In some cases, the bystander approach is combined with the autologous or allogeneic approach. For example, an autologous, allogeneic or bystander cell line encoding a cytokine may be combined with an autologous, allogeneic or bystander cell line. The nucleic acid sequence encoding the cytokine may or may not further comprise a selectable marker sequence operably linked to a promoter. Any combination of cytokines that stimulate an anti-tumor immune response finds utility in the practice of the present invention. The universal bystander cell line preferably grows in defined, i.e., serum-free, medium, preferably as a suspension.

An example of a preferred universal bystander cell line is K562 (ATCC CCL-243; Lozzio et al., Blood 45(3): 321-334 (1975); Klein et al., Int. J. Cancer 18: 421-431 (1976)). A detailed description of human bystander cell lines is described for example in U.S. Pat. No. 6,464,973 and WO9938954. Desirably, the universal bystander cell line expresses the cytokine, e.g., GM-CSF in the range from 200-1000 ng/10⁶ cells/24 h. Preferably, the universal bystander cell line expresses at least about 200 ng GM-CSF/10⁶ cells/24 hours.

In practicing the invention, the one or more universal bystander cell lines are incubated with an autologous cancer antigen, e.g., an autologous tumor cell (which together comprise a universal bystander cell line composition), then the universal bystander cell line composition is administered to the patient. Any suitable route of administration can be used to introduce a universal bystander cell line composition into the patient. Preferably, the composition is administered subcutaneously or intratumorally.

Typically, the cancer antigen is provided by (on) a cell of the cancer to be treated, i.e., an autologous cancer cell. In such cases, the composition is rendered proliferation-incompetent by irradiation, wherein the bystander cells and cancer cells are plated in a tissue culture plate and irradiated at room temperature using a Cs source, as detailed above.

The ratio of bystander cells to autologous cancer cells in a given administration will vary dependent upon the combination. With respect to GM-CSF-producing bystander cells, the ratio of bystander cells to autologous cancer cells in a given administration should be such that at least 36 ng GM-CSF/10⁶ cells/24 hrs is produced. In general, the therapeutic effect is decreased if the concentration of GM-CSF is less than this. In addition to the GM-CSF threshold, the ratio of bystander cells to autologous cancer cells should not be greater than 1:1. Appropriate ratios of bystander cells to tumor cells or tumor antigens can be determined using routine methods in the art.

The use of bystander cell lines in practicing present invention provides the therapeutic advantage that, through administration of a cytokine-expressing bystander cell line and at least one tyrosine kinase inhibitor to a patient with cancer, together with an autologous cancer antigen, paracrine production of an immunomodulatory cytokine, results in an effective immune response to a tumor. This obviates the need to culture and transduce autologous tumor cells for each patient, eliminating the problem of variable and inefficient transduction efficiencies.

Evaluation of Combinations in Animal Models B16F10 Melanoma Model

In one approach, the efficacy of s cytokine-expressing cancer immunotherapy combination is evaluated by carrying out animal studies in the syngeneic B16F10 melanoma tumor model in the treatment setting. See, e.g., Griswold D P Jr., Cancer Chemother Rep 2;3(1):315-24, 1972 and Berkelhammer J et al., Cancer Res 42(8):3157-63, 1982. The murine melanoma cell line B16 is a well-defined cell line which is weakly immunogenic in syngeneic C57BL6 mice and therefore readily forms tumors in C57BL6 mice. Furthermore, several tumor associated antigens have been identified in this model which allow one to monitor tumor as well as antigen specific immune responses. In addition, several murine-specific reagents are commercially available and are used to monitor anti-tumor immune responses in the various immunotherapy vaccine strategies. A typical study in the B16F10 melanoma tumor model makes use of at least 6 and generally 10-15 mice per group in order to obtain statistically significant results. Statistical significance is evaluated using the Student's t-test.

Immunization of C57BL/6 mice with irradiated GM-CSF-secreting B16F10 tumor cells stimulates potent, long-lasting and specific anti-tumor immunity that prevents tumor growth in most mice subsequently challenged with wild-type B16F10 cells. However, this protection is less effective when GM-CSF-producing cancer immunotherapy is administered to mice with preexisting tumor burden. In carrying out studies using the B16F10 melanoma tumor model, female C57BL/6 mice are obtained from Taconic and are 6-8 weeks old at the start of each experiment. In a typical experiment, mice are injected with 1×10⁵ B16BF10 cells on day 0 subcutaneously in a dorsal/anterior location. On day 3, mice are vaccinated in a ventral/posterior location with 1−3×10⁶ irradiated (5000 rads) B16F10 or cytokine-expressing cancer immunotherapy (e.g., GVAX®). Mice are followed for tumor development and survival. After 14-21 days, mice are sacrificed and their tumor burden assessed by harvesting the mice lungs and counting the surface tumor metastasis and measuring the weight of the lung. An alternative B16F10 melanoma tumor model involves subcutaneous injection of B16F10 tumor cells.

A typical in vivo study in the B16F10 melanoma tumor model employs the following groups: HBSS only (negative control); irradiated B16F10/HBSS (control): cytokine-expressing cancer immunotherapy (GVAX®)/HBSS; (cancer immunotherapy monotherapy control); cytokine-expressing cancer immunotherapy plus a tyrosine kinase inhibitor.

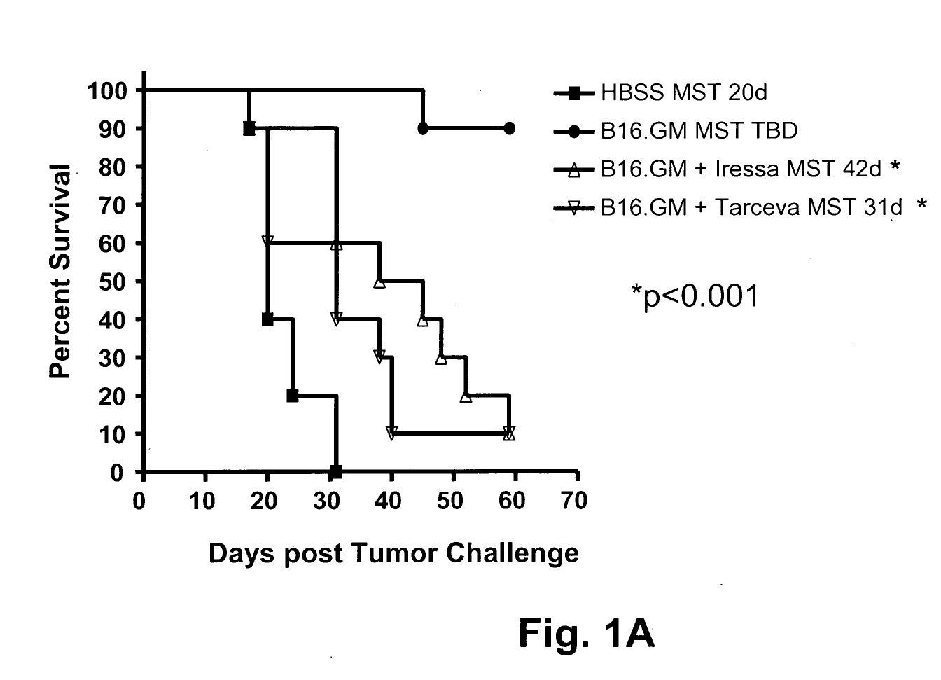

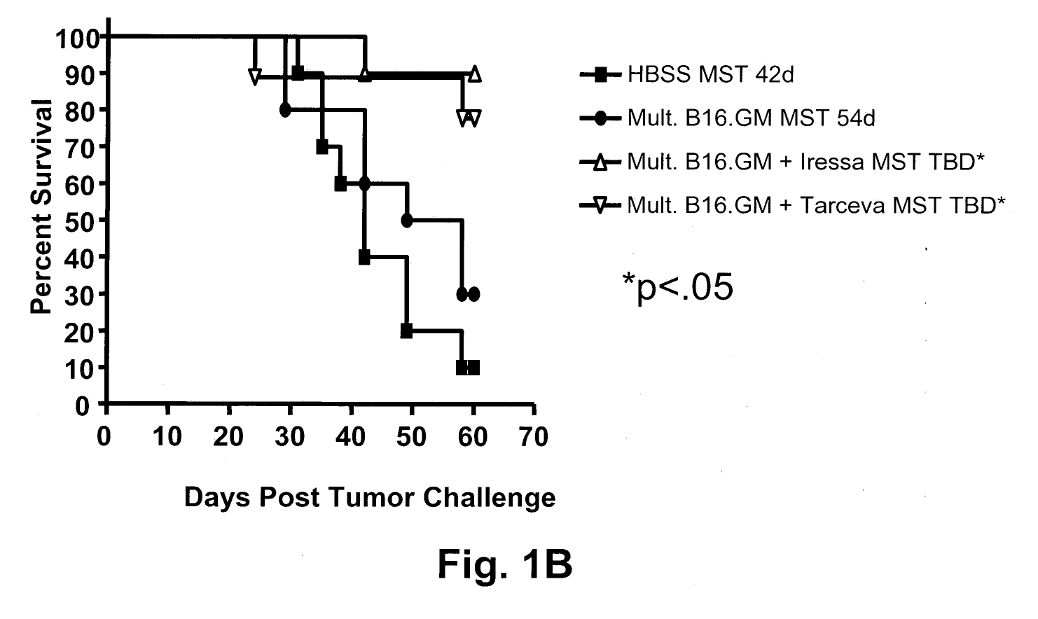

Experiments in the syngeneic B16 melanoma model in C57BL6 mice, have shown that immunity was induced with B16 cells that were genetically modified to express GM-CSF, while non-transduced B16 cells were completely ineffective. Immunization of C57BL6 mice with irradiated B16F10 melanoma cells engineered to secrete GM-CSF has been shown to stimulate potent, long-lasting and specific anti-tumor immunity that prevents tumor formation in a majority of mice challenged with non-transduced B16F10 (prevention model; FIG. 1A). However, when irradiated GM-CSF-producing tumor cells are administered to mice harboring recently established subcutaneous tumors (treatment model; FIG. 1B), the protective anti-tumor immunity is less effective. Results from animal model experiments have convincingly shown that GM-CSF producing tumor cells are able to induce an immune response against the parental, non-transduced tumor cells, even if they are non-immunogenic tumor cells, such as B16F10.

Previous experiments have demonstrated that HBSS or irradiated B16F10 alone do not protect challenged mice from tumor formation. GM-CSF-expressing cancer immunotherapies (GVAX®) alone were shown to protect from 30-50% of the challenged mice in the model. The combination of a cytokine-expressing cancer immunotherapy plus at least one tyrosine kinase inhibitor administered at least 4 days, 7 days, 10 days or 14 days after the cancer immunotherapy is expected to increase the efficacy of anti-tumor protection. The degree of protection depends on several factors such as the expression level of the cytokine-expressing cancer immunotherapy, the level of treatment (i.e. dose of the agent or the frequency and strength of radiation) and the relative timing and route of administration of the tyrosine kinase inhibitor relative to the timing of administration of the cytokine-expressing cancer immunotherapy, e.g., GVAX®.

Immunological Monitoring

Several tumor associated antigens have been identified which allow one to monitor tumor as well as antigen specific immune responses. For example, tumor antigen-specific T cells can be identified by the release of IFN-gamma following antigenic restimulation in vitro (Hu, H-M. et al., Cancer Research, 2002, 62; 3914-3919). Yet another example of methods used to identify tumor antigen-specific T cells is the development of soluble MHC I molecules also known as MHC tetramers (Beckman Coulter, Immunomics), reported to be loaded with specific peptides shown to be involved in an anti-tumor immune response. Examples within the B16F10 melanoma tumor model include but are not limited to gp100, Trp2, Trp-1, and tyrosinase. Similar melanoma-associated antigens have been identified in humans. Such tools provide information that can then be translated into the clinical arena.

B16.OVA Model

B16.ova and B16.GM.ova tumors are B16 cells or B16 cells expressing GM-CSF that were modified to express membrane bound ovalbumin. Ovalbumin acts as a surrogate tumor associated antigen on the tumor cells used for challenge as well as on the cytokine-expressing cancer immunotherapy cells. Ovalbumin-specific T cells are used to track “tumor specific” T cell responses in the presence or absence of cells expressing GM-CSF (B16.GM-ova) alone or in combination with a tyrosine kinase inhibitor. An antibody specifically recognizing the T-cell receptor of the ovalbumin specific T-cells is used to follow these “tumor-specific” T-cells. This antibody can be used to monitor the expansion of these tumor-specific T-cells and their activation status following various immunization and combination regiments. In one exemplary experimental approach, on day-7 mice received daily gavage of a tyrosine kinase inhibitor or vehicle for 20 days. Then ovalbumin-specific T-cells on day-2, were adoptively transferred to mice, then they were challenged with B16F10.ova on day 0, are immunized with B16F10.GM-ova on day 3, followed by monitoring of OVA-specific T cells at various time points after immunization.

Assays for Efficacy of Combinations in Vivo Models

Tumor burden was assessed at various time points after tumor challenge. Typically, spleens cells are assessed for CTL activity by in vitro whole cell stimulation for 5 days. Target cells are labeled with ⁵¹Cr and co-incubated with splenic effector CTL and release of ⁵¹Cr into the supernatants as an indicator of CTL lysis of target cells. On day 3 of in vitro stimulated CTL supernatants are tested for IFN-gamma production by CTL. In brief, wells are coated with coating antibody specific for IFN-gamma, supernatant is then added to wells, and IFN-gamma is detected using an IFN-gamma specific detecting antibody. IFN-gamma can also be detected by flow cytometry, in order to measure cell-specific IFN-gamma production.

Another indication of an effective anti-tumor immune response is the production of effector cytokines such as TNF-alpha, IL-2, and IFN-gamma upon restimulation in vitro. Cytokine levels were measured in supernatants from spleen cells or draining lymph node (dLN) cells restimulated in vitro for 48 hours with irradiated GM-CSF-expressing cells.

A further method used to monitor tumor-specific T cell responses is via intracellular cytokine staining (ICS). ICS can be used to monitor tumor-specific T-cell responses and to identify very low frequencies of antigen-specific T-cells. Because ICS is performed on freshly isolated lymphocytes within 5 hours of removal, unlike the CTL and cytokine release assays, which often require 2-7 days of in vitro stimulation, it can be used to estimate the frequency of tumor antigen-specific T-cells in vivo. This provides a powerful technique to compare the potency of different tumor immunotherapy strategies. ICS has been used to monitor T-cell responses to melanoma-associated antigens such as gp100 and Trp2 following various melanoma immunotherapy strategies. Such T cells can be identified by the induction of intracellular IFN-gamma expression following stimulation with a tumor-specific peptide bound to MHC I.

Xenogen Imaging of Tumor Models

In some studies, in vivo luminescence of tumor bearing mice is monitored by monitoring of B16F10-luciferase (Xenogen Inc.) injected mice. In brief, Balb/c nu/nu mice are injected with 5×10⁴ or 2×10⁵ cells of B16F10-luc cells via tail vein on day 0. Mice are monitored for tumor burden when necessary by intra-peritoneal injection of excess luciferin substrate at 1.5 mg/g mice weight. In a typical analysis, twenty minutes after substrate injection, mice are anesthesized and monitored for in vivo luminescence with Xenogen IVIS Imaging System (Xenogen Inc.) luminescence sensitive CCD camera by dorsal or ventral position. Data are collected and analyzed by Living Image 2.11 software.

Cytokine-Expressing Cancer Immunotherapy Combinations

The present invention is directed to combinations of a cytokine-expressing cancer immunotherapy (e.g., GVAX®) plus at least one tyrosine kinase inhibitor. The tyrosine kinase inhibitor may be any molecule that inhibits the activity of a cellular tyrosine kinase. In one aspect, the at least one tyrosine kinase inhibitor is an anilinoquinazoline, e.g., as exemplified in FIGS. 6A-C. In preferred embodiments, the anilinoquinazoline is an inhibitor of Epidermal Growth Factor Receptor (EGFR) activity (erbB2 kinase) and is selected from the group consisting of genfitinib (Iressa) and erolotinib (Tarceva). Other small molecule tyrosine kinase inhibitors that inhibit EGFR activity include 3-cyanoquinolines (e.g., EKB-569), pyrrlopyrimidines (e.g., PKI-166), and 6-thiazolylquinazolines (e.g., GW572016). In other embodiments, the at least one tyrosine kinase inhibitor is an inhibitor of bcr-abl tyrosine kinase activity, preferably imatinib (Gleevec), or the Flt-3 tyrosine kinase. Other suitable tyrosine kinase inhibitors are reviewed in Traxler Expert Opin. Ther. Targets 7:215-234 (2003).

Anilinoquinazoline and quinazoline derivatives pharmaceutical compositions containing them and the uses thereof based on receptor tyrosine kinase inhibitory properties of the compounds are described for example in U.S. Pat. Nos. 7,001,904; 6,982,270; 6,982,260; 6,911,446; 6,897,214; 6,897,210; 6,541,481; 6,399,602; 6,362,336; 6,015,814; 5,955,464; 5,952,333; 5,942,514; 5,932,574; 5,880,130; 5,866,572; 5,821,246; 5,814,630; 5,770,603; 5,770,599; 5,616,582; 5,580,870; 5,569,658; 5,475,001; and 4,464,375, each of which is expressly incorporated by reference herein in it's entirety.

For instance, genfitinib (Iressa) is clinically accepted as a third line chemotherapy drug for patients with Non Small Cell Lung Cancer (NSCLC). It is a small molecule that inhibits the intracellular phosphorylation of EGF receptor TK, which interferes with downstream signal transduction involved in proliferation, angiogenesis, metastasis, and resistance to apoptosis, thereby leading to cell death. While the anti-angiogenic activity of Iressa will slow/inhibit the growth of a tumor, which will allow cytokine-expressing cancer immunotherapy-induced tumor-specific immune response to destroy the remaining tumor, waiting to administer Iressa until at least 4 days after cytokine-expressing cancer immunotherapy results in an additional benefit of expanding the number of activated, tumor-specific T-cells. Thus, Iressa and a cytokine-expressing cancer immunotherapy such as GVAX® may find utility in the treatment of cancer.

Delivery of Cytokine-Expressing Cancer Immunotherapy to the Patient

The present invention provides methods for cancer therapy, where a cytokine-expressing cancer immunotherapy and at least one tyrosine kinase inhibitor are administered to a cancer patient. Desirably, the method effects a systemic immune response, i.e., a T-cell response and/or a B-cell response, to the cancer.

In a preferred aspect of the methods described herein, a cytokine-expressing cancer immunotherapy combination is administered to a cancer patient, wherein the cytokine-expressing cancer immunotherapy comprises mammalian, preferably human tumor cells, and the cells in the cytokine-expressing cancer immunotherapy are rendered proliferation incompetent, such as by irradiation. Administration of a cytokine-expressing cancer immunotherapy combination results in an enhanced immune response to the cancer as compared to the immune response to the same cancer following administration of the cytokine-expressing cancer immunotherapy or tyrosine kinase inhibitor component of the combination alone. In other words, the combined administration of a cytokine-expressing cancer immunotherapy and at least one additional cancer therapeutic agent or treatment described above results in enhanced therapeutic efficacy as compared to administration of a cytokine-expressing cancer immunotherapy alone or administration of the at least one tyrosine kinase inhibitor alone.

The cytokine-expressing cancer immunotherapy combination may be administered by any suitable route. Preferably, the composition is administered subcutaneously or intratumorally. Local or systemic delivery can be accomplished by administration comprising administration of the combination into body cavities, by parenteral introduction, comprising intramuscular, intravenous, intraportal, intrahepatic, peritoneal, subcutaneous, or intradermal administration. In the event that the tumor is in the central nervous system, the composition is administered in the periphery to prime naïve T-cells in the draining lymph nodes. The activated tumor-specific T-cells are able to cross the blood/brain barrier to find their targets within the central nervous system.

In one exemplary preferred embodiment, the cytokine-expressing cancer immunotherapy is GVAX®, where the cytokine expressed is GM-CSF and at least one tyrosine kinase inhibitor is an anilinoquinazoline that inhibits the activity of Epidermal Growth Factor Receptor (EGFR) activity (erbB2 kinase) and is selected from the group consisting of genfitinib (Iressa) and erolotinib (Tarceva). In other embodiments, the at least one tyrosine kinase inhibitor is an inhibitor of bcr-abl tyrosine kinase activity, preferably imatinib (Gleevec).

As will be understood by those of skill in the art, the optimal treatment regimen will vary. As a result, it will be understood that the status of the cancer patient and the general health of the patient prior to, during, and following administration of a cytokine-expressing cancer immunotherapy combination, the patient will be evaluated in order to determine if the dose of each component and relative timing of administration should be optimized to enhance efficacy or additional cycles of administration are indicated. Such evaluation is typically carried out using tests employed by those of skill in the art to evaluate traditional cancer chemotherapy, as further described below in the section entitled “Monitoring Treatment”.

Delivery of Tyrosine Kinase Inhibitors

In an aspect of the invention, the cytokine-expressing cancer immunotherapy combination comprises a tyrosine kinase inhibitor. An important consideration in this aspect of the invention is effective delivery of the tyrosine kinase inhibitor in a pharmaceutically acceptable carrier.

In accordance with this aspect of the invention, the choice of tyrosine kinase inhibitor and the corresponding route and timing of delivery takes advantage of one of more of: (i) established use in treatment of the particular type of cancer under treatment; (ii) the ability of the selected agent to result in an improved therapeutic outcome when administered in combination with the cytokine-expressing cancer immunotherapy; and (iii) delivery of the agent by a mode of administration effective to achieve sufficient localized exposure of the agent to cancer cells.

Typically, the tyrosine kinase inhibitor is administered by a route and using a treatment regimen that has an established use in cancer therapy. As set forth above, the optimal route will vary with the tyrosine kinase inhibitor. Local or systemic delivery can be accomplished by administration into body cavities, inhalation or insufflation of an aerosol, or by parenteral introduction, comprising intramuscular, intravenous, intraportal, intrahepatic, peritoneal, subcutaneous, or intradermal administration. However, preferred routes typically include slow intravenous infusion (IV drip), oral administration and local injection. In the event that the tumor is in the central nervous system, the composition must be administered into the periphery via any route. The formulations are easily administered in a variety of dosage forms such as injectable solutions, drug release capsules, implants or in combination with carriers such as liposomes or microcapsules.

Parenteral administration may be accomplished using a suitable buffered aqueous solution and the liquid diluent which has been prepared in isotonic form using saline or glucose. Such aqueous solutions are appropriate for intravenous, intramuscular, subcutaneous and intraperitoneal administration. (See, for example, “Remington's Pharmaceutical Sciences”, 15th Edition, pages 1035-1038 and 1570-1580.) Sterile injectable solutions are prepared by incorporating the chemotherapeutic agent in the required amount of an appropriate solvent with various other ingredients included, followed by filter sterilization. Sterile powders for use in sterile injectable solutions may be prepared by vacuum drying or freeze drying techniques or other means to result in a powder of the active tyrosine kinase inhibitor plus additional desired ingredients prepared from a previously sterile solution.

For example, when orally administered, the tyrosine kinase inhibitor may be combined with an inert diluent or in an edible carrier, or enclosed in hard or soft shell gelatin capsules, compressed into tablets, incorporated directly into food, incorporated with excipients and used in the form of ingestible tablets, buccal tables, troches, capsules, elixirs, suspensions, syrups, wafers, and the like. The appropriate amount of tyrosine kinase inhibitor is specific to the particular tyrosine kinase inhibitor and is generally known in the art.

Recommended dosages and dosage forms for a large number of cancer therapeutic agents have been established and can be obtained from conventional sources, such as the Physicians Desk Reference, published by Medical Economics Company, Inc., Oradell, N.J. Typically, the optimal route of delivery has been determined for known cancer therapeutic agents by well-established procedures and analysis, e.g., in clinical trials.

It will be understood that the invention contemplates treatment regimens that include the administration of at least one tyrosine kinase inhibitor and administration of a cytokine-expressing cancer immunotherapy for therapy of cancer. Such a treatment regimen may be administered prior to, contemporaneously with, or subsequent to an additional cancer treatment, such as radiation therapy, further chemotherapy and/or immunotherapy. In a preferred embodiment, the tyrosine kinase inhibitor is an inhibitor of Epidermal Growth Factor Receptor (EGFR) activity, more preferably is an anilinoazoquinoline, and is administered to the subject about 4 days, 7 days, 10 days or 14 days following administration of the cytokine-expressing cancer immunotherapy component of the combination to enhance the proliferation of activated T-cells thereby enhancing the efficacy of the cytokine-expressing cancer immunotherapy.

The present invention provides the advantage that the dose of the tyrosine kinase inhibitor may be decreased when administered together with a cytokine-expressing cancer immunotherapy relative to treatment regimens that do not include cytokine-expressing cancer immunotherapy administration.

Monitoring Treatment

One skilled in the art is aware of means to monitor the therapeutic outcome and/or the systemic immune response upon administering a combination treatment of the present invention. In particular, the therapeutic outcome may be assessed by monitoring the attenuation of tumor growth and/or tumor regression and/or the level of tumor specific markers. The attenuation of tumor growth or tumor regression in response to treatment can be monitored using several end-points known to those skilled in the art including, for instance, number of tumors, tumor mass or size, or reduction/prevention of metastasis.

All literature and patent references cited above are hereby expressly incorporated by reference herein.

### EXAMPLE 1

Administration of a Tyrosine Kinase Inhibitor Post Administration of a Cytokine-Expressing Cancer Immunotherapy Enhances Survival in a Tumor Prevention Animal Model

The timing of co-administration of a cytokine-expressing cancer immunotherapy and a tyrosine kinase inhibitor on survival in tumor bearing animals was examined using a tumor prevention animal model system. Mice were pretreated daily by oral gavage with 200 mg/kg of genfitinib (Iressa) or erolotinib (Tarceva) starting on Day-11, which is less than a therapeutically effective dose as a monotherapy in order to observe the effect of the cancer immunotherapy combination. On Day-7, mice were immunized (subcutaneously; SC) with 1×10⁶ irradiated B16F10 tumor cells engineered to express GM-CSF. Seven days later, mice were challenged (SC) with 1×10⁶ live B16F10 tumor cells and followed for tumor development and survival. A Kaplan-Meir survival graph of the results of this study indicates that post- or concurrent administration of a cytokine-expressing cancer immunotherapy and a tyrosine kinase inhibitor results in decreased survival in a basic prevention model (FIG. 1A).

The timing of co-administration of a cytokine-expressing cancer immunotherapy and a tyrosine kinase inhibitor on survival in tumor bearing animals was also examined in a basic treatment model. In this model, mice were inoculated (SC) with 2×10⁵ live B16F10 tumor cells mice on Day 0 and immunized (SC) with 1×10⁶ irradiated B16F10 tumor cells engineered to express GM-CSF on Days 3 and 17. On Day 15, mice were treated daily by oral gavage with 200 mg/kg of genfitinib (Iressa) or erolotinib (Tarceva), and followed for tumor development and survival. A Kaplan-Meir survival graph of the results of a study in C57Bl/6 mice indicating administration of a cytokine-expressing cancer immunotherapy prior to administering a tyrosine kinase inhibitor results in increased survival (FIG. 1B).

Therefore, the timing of the administration of the tyrosine kinase inhibitor, both anilinoquinazolines, relative to cancer immunotherapy administration was critical to observed enhanced efficacy of the cancer immunotherapy. It appears that prior or concurrent administration of the tyrosine kinase inhibitor has a deleterious effect on the therapeutic efficacy of the cancer immunotherapy.

### EXAMPLE 2

Tyrosine Kinase Inhibitors Block the Priming of Naïve T-cells but Increase the Number of Activated T-cells

To examine the effect of tyrosine kinase inhibitors on naïve T-cell priming, C57Bl6 mice were inoculated with 5×10⁵ B16F10 tumor cells transduced to express ovalbumin as a surrogate antigen on Day 0. One day later, mice began receiving daily by oral gavage 200 mg/kg of genfitinib (Iressa) or erolotinib (Tarceva). Three days later, mice were immunized with 1×10 ⁶ irradiated GM-CSF-secreting B16F10 cells engineered to express ovalbumin. Fourteen days later, mice were sacrificed and the spleens were removed and evaluated for induction of a primary T-cell response by quantifying the number of IFN-gamma-secreting T-cells per 5×10⁵ splenocytes when stimulated with an ovalbumin-specific peptide (SIINFEKL).

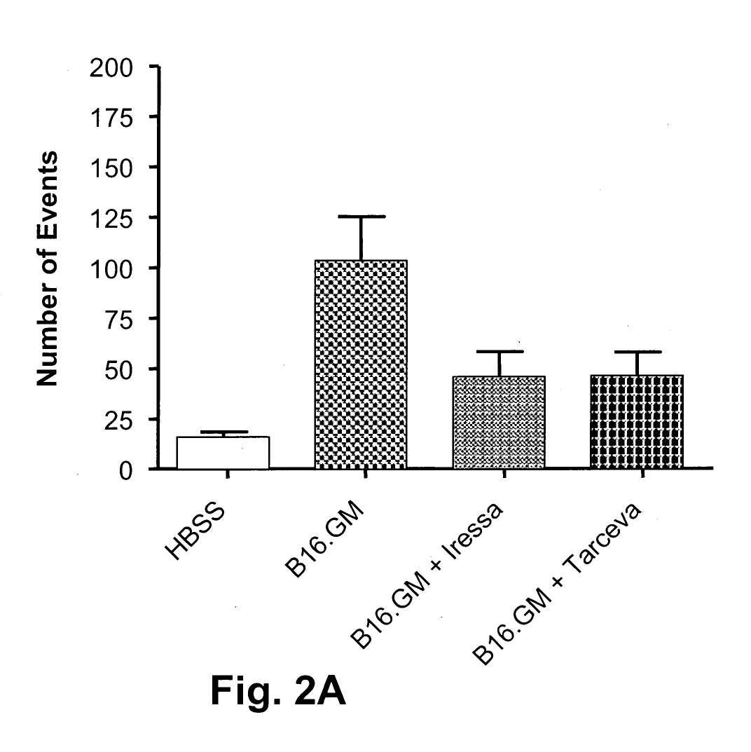

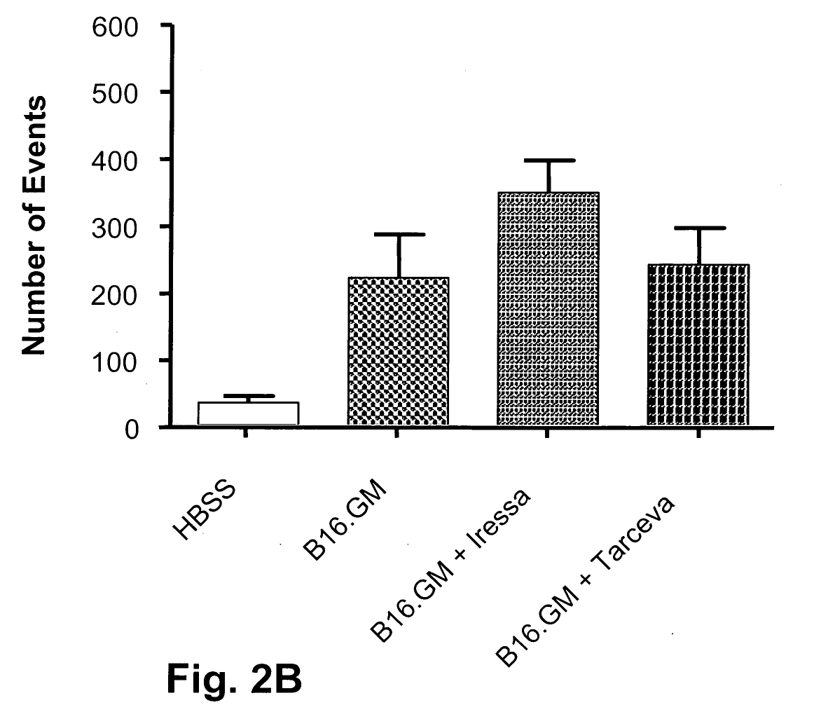

As shown in FIG. 2A, administration of genfitinib (Iressa) or erolotinib (Tarceva) prior to and/or concurrently with the cytokine-expressing cancer immunotherapy resulted in a decrease in the number of primed naïve T-cells. Since antigen priming of naïve T-cells and subsequent activation and proliferation of such T-cells is believed to be an important aspect for an efficacious cancer immunotherapy, these results may explain the decreased survival observed in the tumor prevention animal model in Example 1 due to a reduced cytotoxic T-cell response against the tumor.

To examine the effect of tyrosine kinase inhibitors on activation of primed T-cells, C57Bl6 mice were inoculated with 5×10⁵ B16F10 tumor cells transduced to express ovalbumin as a surrogate antigen on Day 0. On Days 3 and 17, mice were immunized with 1×10⁶ irradiated GM-CSF-secreting B16F10 cells engineered to express ovalbumin. On Day 15, mice began receiving daily by oral gavage 200 mg/kg of genfitinib (Iressa) or erolotinib (Tarceva). Mice were sacrificed on Day 31 and the spleens were removed and evaluated for induction of a primary T-cell response by quantifying the number of IFN-gamma-secreting T-cells per 5×10⁵ splenocytes when stimulated using an ovalbumin-specific peptide.

As shown in FIG. 2B, administration of genfitinib (Iressa) or erolotinib (Tarceva) post administration of the cytokine-expressing cancer immunotherapy resulted in a slight increase in the number of ova-antigen activated T-cells. Since activation and proliferation of anti-tumor antigen primed T-cells is believed to be an important aspect for an efficacious cancer immunotherapy, it is possible that the increased survival observed in the tumor treatment animal model in Example 1 is associated with an increased cytotoxic T-cell response against the tumor.

### EXAMPLE 3

Tyrosine Kinase Inhibitors Block Murine and Human Naïve T-cell Activation and Proliferation

C57Bl/6 mouse lymphocytes or human PBMCs were stimulated using anti-CD3 or ConA, respectively. For mouse T cell proliferation, equal numbers of mouse lymphocytes and irradiated antigen presenting cells (1.5×10⁶ cells) were co-cultured for 72 hours in the presence of serially diluted genfitinib (Iressa) or erolotinib (Tarceva) at concentrations ranging from 100 mg/ml to 100 pg/ml. Human T-cell proliferation assay was conducted similarly using 1.5×10⁶ PBMCs co-cultured with 1.5×10⁶ irradiated APCs for 72 hours in the presence of serially diluted genfitinib (Iressa) or erolotinib (Tarceva) at concentrations 100 mg/ml to 100 pg/ml. Cellular proliferation was measured by the addition of 1 mCi 3H-thymidine during the last 6 hours of culture. Cells were harvested and counted using a beta counter. Percent inhibition was determined relative to cells alone (no inhibition).

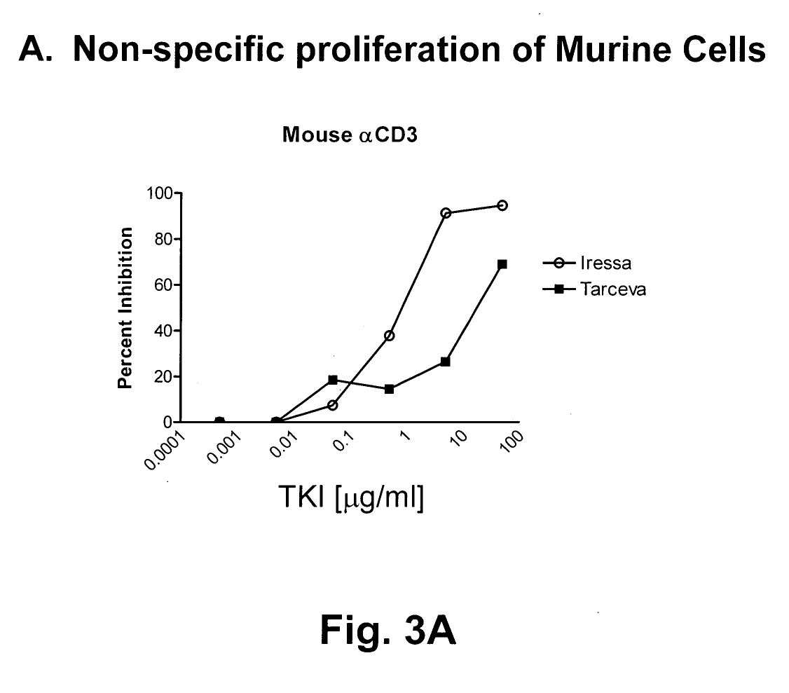

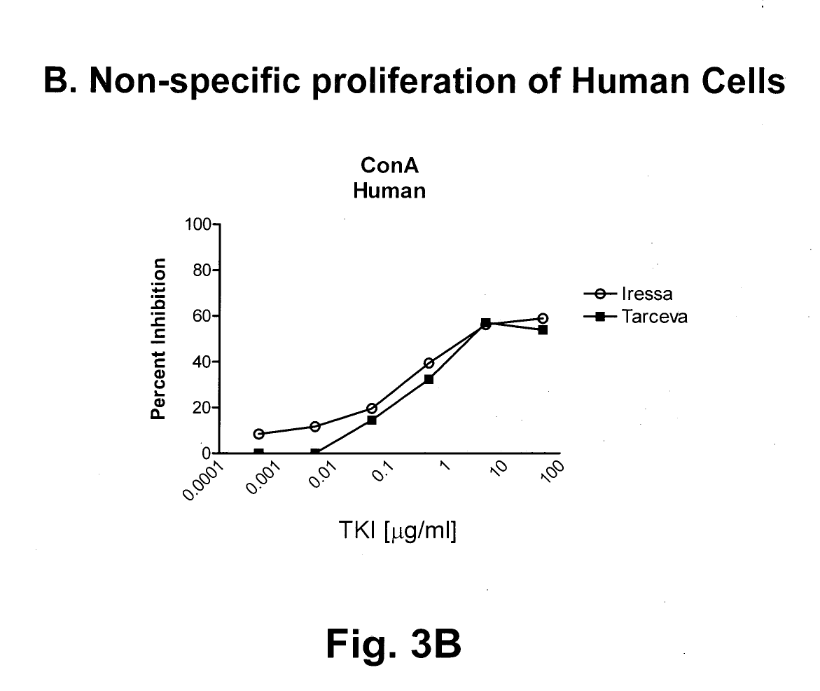

As shown in FIG. 3, increasing concentrations of the anilinoquinazoline tyrosine kinase inhibitors resulted in decreased relative percentage of activated T-cells as shown for the murine C57BL/6 lymphocytes stimulated with anti-CD3 antibody (FIG. 3, panel A) as well as the human PBMC stimulated with ConA (FIG. 3, panel B).

### EXAMPLE 4

Tyrosine Kinase Inhibitors Block the Phosphorylation of Tyrosine Kinases Involved in T-cell Activation

Nutrients-starved human T cell clones (Jurkat cells) were stimulated for 10 minutes using 10 mcg/ml of human anti-CD3 antibodies in the presence of vehicle control (DMSO) or 0, 12.5, or 50 mcg/ml of genfitinib (Iressa) or erolotinib (Tarceva). Cells were lysed using RIPA buffer and immuno-precipitated using human anti-Pyk2, anti-Zap-70 or anti-Lck. Proteins were boiled, separated by electrophoeresis using a 4%-12% gradient SDS-PAGE gel and transferred to nitrocellulose membrane. Membranes were immuno-blotted using a human anti-phospho-Pyk2, anti-phospho-Zap-70 or anti-phospho-Lck antibody and visualized using a labeled secondary antibody.

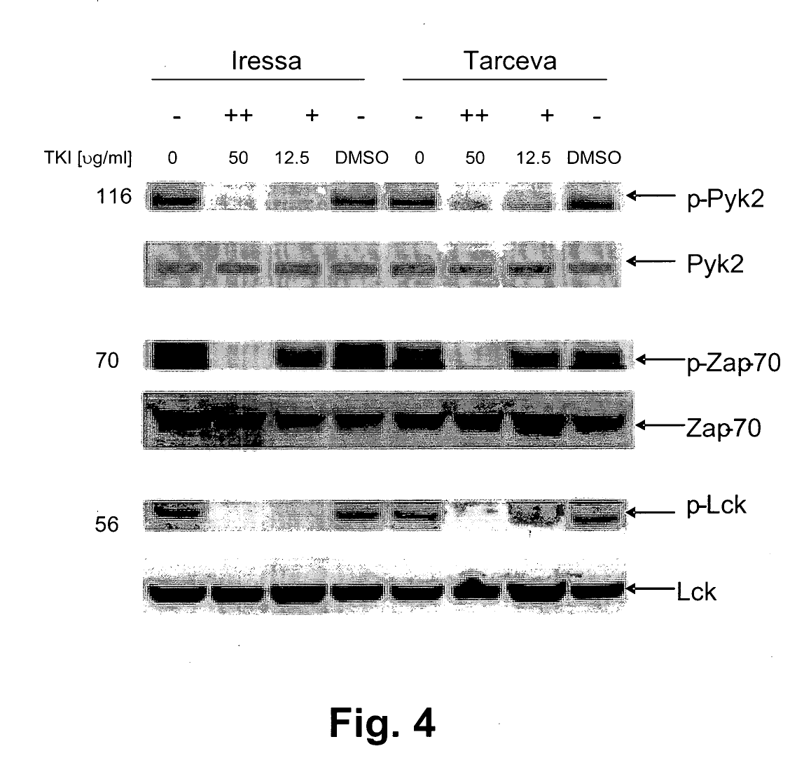

The results of the Western blot evaluating the presence of phosphorylated and non-phosphorrylated tyrosine kinases of stimulated Jurkat cells when co-incubated with a dose titration of genfitinib (Iressa) or erolotinib (Tarceva) is shown in FIG. 4. Increasing concentrations of genfitinib (Iressa) or erolotinib (Tarceva) resulted in a concomitant decreased in the amount of phosphorylated Pyk2, Zap-70 or anti-Lck proteins, which are known to play a role in the activation of T-cells.

These results are consistent with the decreased survival observed in the tumor prevention animal model in Example 1 due to a reduced number of activate, cytotoxic T-cell targeted against the tumor.

### EXAMPLE 5

Tyrosine Kinase Inhibitors Augment the Expansion of Adoptively Transferred Ovalbumin Transgenic T-cells upon Immunization with a Cytokine-Expressing Cancer Immunotherapy

To examine whether tyrosine kinase inhibitors augment the expansion of adoptively transferred transgenic T cells, indicated C57BL/6 recipient mice received daily oral gavages of 200 mg/kg genfitinib (Iressa) or erolotinib (Tarceva) or vehicle beginning on Day-7 only for 20 days. On Day-2, 4×10⁶ splenocytes from OT-1 transgenic mice were adoptively transferred into recipient mice. On Day 0, mice were challenged with 2×10⁵ live B16F10 cells transduced to express the surrogate antigen, ovalbumin and subsequently immunized with 1×10⁶ irradiated GM-CSF-secreting B16F10 cells transduced to express ovalbumin (GM.ova) on day 3. On Day 9, spleens and lymph nodes were harvested, double stained with OT-1-specific tetramers and CD8-specific antibody and evaluated by FACs analysis.

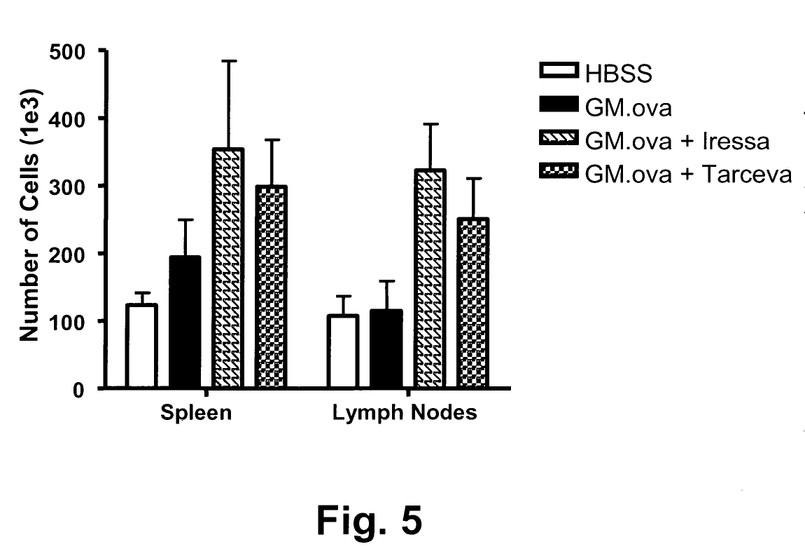

The results of this experiment are shown in FIG. 5. In the presence of genfitinib (Iressa) or erolotinib (Tarceva), the absolute number of ovalbumin-specific CD8 T cells in spleen and lymph nodes is increased relative to administration of the cytokine-expressing cancer immunotherapy alone. This result is consistent with those presented above that demonstrate that provided that the T-cells have been sufficiently primed prior to exposure to the tyrosine kinase inhibitor, an increase in the relative numbers of activated T-cells and prolonged survival is observed in the animal receiving the cytokine-expressing cancer immunotherapy prior to administration of the tyrosine kinase inhibitor.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes

It is claimed:

1. An improved method for cancer immunotherapy therapy, comprising:
administering a cytokine-expressing cancer immunotherapy composition to a subject with cancer;
allowing for a sufficient amount of time for activation of anti-tumor specific T-cells; and
administering at least one tyrosine kinase inhibitor to said subject;
wherein following administration of said immunotherapy composition and said at least one tyrosine kinase inhibitor, the subject exhibits an enhanced therapeutic efficacy relative to the therapeutic effect exhibited following administration of the cytokine-expressing cancer immunotherapy or the at least one tyrosine kinase inhibitor alone.

2. The method of claim 1, wherein the cytokine-expressing cancer immunotherapy composition comprises cells that express granulocyte-macrophage colony stimulating factor (GM-CSF).

3. The method of claim 2, wherein the cells of said cytokine-expressing cancer immunotherapy composition are autologous to the subject.

4. The method of claim 2, wherein the cells of said cytokine-expressing cancer immunotherapy composition are allogeneic to the subject.

5. The method of claim 2, wherein the cells of said cytokine-expressing cancer immunotherapy composition are bystander cells.

6. The method of claim 2, wherein the cells of the cytokine-expressing cancer immunotherapy composition are rendered proliferation-incompetent by irradiation.

7. The method of claim 2, wherein the mammal is a human.

8. The method of claim 2, wherein the cancer is a prostate cancer.

9. The method of claim 2, wherein the cancer is a non-small cell lung carcinoma.

10. The method of claim 4, wherein the allogeneic cells are a tumor cell line selected from the group consisting of a prostate tumor line, a non-small cell lung carcinoma line and a pancreatic cancer line.

11. The method of claim 2, wherein said at least one additional cancer therapeutic agent is expressed by a cell and the cell is an autologous, allogeneic or a bystander cell.

12. The method of claim 11, wherein the autologous, allogeneic or a bystander cell is rendered proliferation-incompetent by irradiation.

13. The method of claim 2, wherein said cytokine-expressing cancer immunotherapy composition is administered subcutaneously.

14. The method of claim 2, wherein said cytokine-expressing cancer immunotherapy composition is administered intratumorally.

15. The method of claim 2, wherein said at least one tyrosine kinase inhibitor is an anilinoquinazoline tyrosine kinase inhibitor.

16. The method of claim 15, wherein said anilinoquinazoline tyrosine kinase inhibitor is gefitinib.

17. The method of claim 15, wherein said anilinoquinazoline tyrosine kinase inhibitor is erolotinib.

18. The method of claim 12, wherein said at least one tyrosine kinase inhibitor is an anilinoquinazoline tyrosine kinase inhibitor.

19. The method of claim 18, wherein said anilinoquinazoline tyrosine kinase inhibitor is gefitinib.

20. The method of claim 18, wherein said anilinoquinazoline tyrosine kinase inhibitor is erolotinib.

21. The method of claim 2, wherein said tyrosine kinase inhibitor is imatinib.

22. The method of claim 12, wherein said tyrosine kinase inhibitor is imatinib.

23. The method of claim 2, wherein said tyrosine kinase inhibitor is administered to the subject about 4 days, 7 days, 10 days or 14 days following administration of the cytokine-expressing cancer immunotherapy composition.

24. A method for enhancing the therapeutic benefit of a cancer immunotherapy comprising;
administering a cytokine-expressing cancer immunotherapy composition to a subject with cancer;
allowing for a sufficient amount of time for activation of anti-tumor specific T-cells; and
administering at least one tyrosine kinase inhibitor to the subject;
whereby following administration of said immunotherapy composition and said at least one tyrosine kinase inhibitor, an increase in the number and/or proliferation of activated T-cells is detected relative to the number and/or proliferation of activated T-cells detected following administration of the cytokine-expressing cancer immunotherapy alone.

25. The method of claim 24, wherein the cytokine-expressing cancer immunotherapy composition expresses GM-CSF.

26. The method of claim 25, wherein the cells of said cytokine-expressing cancer immunotherapy are autologous to the subject.

27. The method of claim 25, wherein the cells of said cytokine-expressing cancer immunotherapy are allogeneic to the subject.

28. The method of claim 25, wherein the cells of said cytokine-expressing cancer immunotherapy cells are bystander cells.

29. The method of claim 25, wherein the cells of said cytokine-expressing cancer immunotherapy are rendered proliferation-incompetent by irradiation.

30. The method of claim 27, wherein said allogeneic cells are a tumor cell line selected from the group consisting of a prostate tumor line, a non-small cell lung carcinoma Jine and a pancreatic cancer line.

31. The method of claim 29, wherein said at least one tyrosine kinase inhibitor is an anilinoquinazoline tyrosine kinase inhibitor.

32. The method of claim 31, wherein said anilinoquinazoline tyrosine kinase inhibitor is gefitinib.

33. The method of claim 31, wherein said anilinoquinazoline tyrosine kinase inhibitor is erolotinib.
